(19) United States
(12) Patent Application Publication
(10) Pub. No.: US 2019/0201444 A1
(43) Pub. Date: Jul. 4, 2019

(54) GLYCOENGINEERING OF E-SELECTIN LIGANDS

(71) Applicant: Robert Sackstein, Sudbury, MA (US)

(72) Inventor: Robert Sackstein, Sudbury, MA (US)

(21) Appl. No.: 16/303,185

(22) PCT Filed: May 22, 2017
(86) PCT No.: PCT/US17/33868

§ 371 Date: Nov. 20, 2018

Related U.S. Application Data
(60) Provisional application No. 62/339,704, filed on May 20, 2016.
(60) Provisional application No. 62/354,350, filed on Jun. 24, 2016.

(51) Int. Cl. A61K 35/17; A61K 9/00; A61K 35/28; C12N 5/0775; A61P 35/00

(52) U.S. Cl. CPC A61K 35/17; A61K 9/0019; C12Y 204/01214; C12N 5/0663; A61P 35/00; A61K 35/28

(57) ABSTRACT

The present invention provides methods of enforcing expression of an E-selectin and/or L-selectin ligand on a surface of a cell. Also provided are methods of enabling and/or increasing binding of a cell to E-selectin and/or L-selectin, methods of increasing homing and/or extravasation in a population of transplanted cells, methods of producing modified cells, including stem cells, for transplanting into a subject, methods of treating or ameliorating the effects of a symptom, a disease or an injury in a subject, and methods for inducing and/or enhancing homing of a population of cells to a therapeutic target in a subject. The invention further provides pharmaceutical compositions comprising a population of cells produced by the methods of the invention and kits that include such compositions for treating or ameliorating the effects of a symptom, a disease or an injury in a subject.

### CROSS-REFERENCE TO RELATED APPLICATIONS

The present application claims priority to U.S. Provisional Patent Application No. 62/339,704, filed on May 20, 2016, and U.S. Provisional Patent Application No. 62/354,350, filed on Jun. 24, 2016. The entire contents of the aforementioned applications are incorporated by reference as if recited in full herein.

### BACKGROUND OF THE INVENTION

Mesenchymal stem cells (MSCs) hold much promise for cell therapy due to their convenient isolation and amplification in vitro, multi-lineage differentiation ability, tissue-repairing trophic effects, and potent immunomodulatory capacity [Dominici 2006, Griffin 2013]. In particular, because MSCs are precursors of bone-forming osteoblasts, these cells have drawn great interest for treatment of systemic bone diseases such as osteoporosis or osteogenesis imperfecta. However, to achieve this goal, it is first necessary to optimize osteotropism of intravascularly administered MSCs.

Recruitment of circulating cells to bone is dependent on E-selectin receptor/ligand adhesive interactions. E-selectin is a calcium-dependent lectin that is expressed constitutively on marrow microvessels, and inducibly expressed on microvessels at inflammatory sites [Sipkins 2005, Schweitzer 1996, Sackstein 2009]. E-selectin prototypically binds a sialofucosylated terminal tetrasaccharide motif known as sialyl Lewis X (sLe^{X}; NeuAc-α(2,3)-Gal-β(1,4)-[Fuc-α(1,3)]GlcNAc-R). sLe^{X} can be displayed at the terminal end of glycan chains that modify specific cell surface glycoproteins such as PSGL-1, CD43, or CD44. When sLe^{X} is displayed by these proteins, they can function as the E-selectin ligands CLA, CD43E or HCELL, respectively [Dimitroff 2001, Sackstein 2008]. These structures are expressed at high levels on hematopoietic stem and progenitor cells (HSPCs) and other hematopoietic cells, but are completely absent on MSCs. In part due to this deficiency of E-selectin ligands, only a small fraction of injected MSCs home to the bones upon intravenous transplantation [Schrepfer 2007, Lee 2009, Ankrum 2010].

The glycan modifications necessary to create E-selectin ligands are performed in the Golgi by specific glycosyltransferases acting in a stepwise fashion. Human MSCs express high levels of CD44, as well as glycosyltransferases required for synthesis of sLe^{X}, with the notable exception being a complete lack of expression of any of the fucosyltransferases that mediate alpha-(1,3)-fucosylation: FTIII, FTIV, FTV, FTVI, or FTVII [Sackstein 2009]. As such, MSCs express CD44 at the cell surface that is decorated with terminal sialylated lactosamines (NeuAc-α(2,3)-Gal-β(1,4)-GlcNAc-R), requiring only the addition of an alpha-(1,3)-fucose to be converted into the potent E-selectin ligand HCELL. Previously, we developed a method to modify glycans on the surface of MSCs to create E-selectin ligands by incubating intact cells with purified alpha-(1,3)-fucosyltransferase enzyme FTVI and its nucleotide sugar donor GDP-fucose. This method, termed ‘glycosyltransferase mediated stereosubstitution’ (GPS), results in the temporary creation of E-selectin ligands (primarily HCELL) on the MSC cell surface. Such FTVI-driven exofucosylation of MSCs has been demonstrated to robustly enhance E-selectin-mediated tethering and rolling on endothelial cells, and, in preclinical studies, has engendered MSC osteotropism (i.e., homing to bone) [Sackstein 2008]. Based in part on these results, the efficacy of this approach is now being investigated in a clinical trial using exofucosylated MSCs for treatment of osteoporosis [NCT02566655, clinicaltrials.gov].

### SUMMARY OF THE INVENTION

Despite the promise of these methods, there exists an ongoing need for improved methods of engineering cell surface proteins, such as E-selectin ligands, that provide robust modification, homing and engraftment necessary for cell therapy. In part, the present invention provides an alternative approach, in which fucosyltransferase enzyme can be generated intracellularly by introducing synthetic modified mRNA (modRNA) [Levy 2013, Warren 2010]. Similar to exofucosylation, the resultant effects are temporary, enabling the MSCs to return to their natural state after homing. However, the modRNA approach is distinct because it utilizes the MSC's own cellular machinery to produce the fucosyltransferase enzyme, with access to intracellular stores of GDP-Fucose. Furthermore, endogenous FTVI is membrane-bound and anchored in the Golgi membrane, while purified FTVI used for exofucosylation is soluble, consisting of only the stem and catalytic domains of the protein. Unresolved biological questions about the modRNA approach remain, especially since the Golgi localization could enable enzyme access to acceptors that differ from those accessible to fucosylation on the cell surface. As such, it is unknown whether the E-selectin ligands created by exofucosylation are similar in identity and function to those that would be created by the action of intracellular fucosyltransferase. Furthermore, the kinetics by which newly synthesized E-selectin ligands are displayed on (and subsequently disappear from) the MSC surface are likely different from that of exofucosylated MSCs. Most importantly, it is not known whether such differences would lead to dissimilarity in the E-selectin ligand-mediated functional abilities of these cells to home to bone marrow.

To address these questions, we undertook a direct comparison between intracellular and extracellular fucosylation using the same alpha-(1,3)-fucosyltransferase in a human cell natively devoid of such enzymes. To this end, using multiple primary cultures of human MSCs, we utilized modRNA to transiently produce FTVI protein in human MSCs, and compared the biochemical and functional properties of the resulting E-selectin ligands with those created via FTVI exofucosylation. Furthermore, we directly compared the in vivo homing properties of both types of treated cells by performing in vivo imaging of transplanted MSCs in mouse calvarium. This in-depth comparison of FTVI-mediated intracellular versus extracellular fucosylation provides critical information on the activity and function of fucosyltransferase VI in programming cell migration, providing key insights regarding the most appropriate fucosylation approach for clinical utility.

Accordingly, the present invention provides methods of enforcing expression of an E-selectin and/or L-selectin ligand on a surface of a cell, the method comprising the steps of: providing to the cell a nucleic acid encoding a glycosyltransferase, and culturing the cell under conditions sufficient to express the glycosyltransferase, wherein the expressed glycosyltransferase modifies a terminal sialylated lactosamine present on a glycoprotein of the cell to enforce expression the E-selectin and/or L-selectin ligand.

The present invention also provides methods of enabling and/or increasing binding of a cell to E-selectin and/or L-selectin, the method comprising the steps of: providing to the cell a nucleic acid encoding an alpha 1,3-fucosyltransferase, and culturing the cell under conditions sufficient for expression of the alpha 1,3-fucosyltransferase by the cell, wherein the alpha 1,3-fucosyltransferase modifies a glycan chain present on a glycoprotein to create an E-selectin and/or L-selectin ligand and thereby enable and/or increase the binding of the cell to E-selectin and/or L-selectin.

In other embodiments, the present invention provides a method of increasing homing and/or extravasation in a population of cells transplanted into a subject, the method comprising the steps of: providing to the population of cells a nucleic acid encoding an alpha 1,3-fucosyltransferase, culturing the population of cells under conditions sufficient for expression of the alpha 1,3-fucosyltransferase by one or more modified cells within the population, wherein the alpha 1,3-fucosyltransferase fucosylates a glycan chain present on a glycoprotein to create modified cells in which E-selectin and/or L-selectin ligand expression is enforced; and transplanting the population of cells into the subject, wherein the modified cells having enforced E-selectin and/or L-selectin ligand expression display increased homing and/or extravasation to therapeutically useful sites.

The present invention also provides methods of producing modified cells for transplanting into a subject in need thereof, the method comprising the steps of: obtaining a population of cells to be modified, providing to the population of cells a nucleic acid encoding an alpha 1,3-fucosyltransferase, culturing the population of cells under conditions sufficient for expression of the alpha 1,3-fucosyltransferase by one or more modified cells within the population; wherein the alpha 1,3-fucosyltransferase modifies a glycan chain present on a glycoprotein to create an E-selectin and/or L-selectin ligand.

The present invention also provides methods of producing modified stem cells for transplanting into a subject, the method comprising the steps of: obtaining a population of stem cells to be modified; providing to the population of stem cells a cDNA or modified RNA encoding an alpha 1,3-fucosyltransferase; and culturing the population of stem cells under conditions sufficient for expression of the alpha 1,3-fucosyltransferase by one or more modified cells within the population, wherein the expressed alpha 1,3-fucosyltransferase fucosylates CD44 present on or in the one or more modified cells.

The present invention also provides methods of treating or ameliorating the effects of a symptom, a disease or an injury in a subject in need thereof, the method comprising the steps of: obtaining a population of cells produced by any of the methods of the invention, and transplanting an effective amount of the population of cells into the subject; wherein the transplanted cells extravasate to a site expressing E-selectin and/or L-selectin so as thereby to treat or ameliorate the effects of the symptom, disease or injury in the subject.

The present invention also provides pharmaceutical compositions comprising a population of cells produced by the methods of the invention and a pharmaceutically acceptable carrier.

The present invention also provides kits for treating or ameliorating the effects of a symptom, a disease or an injury in a subject in need thereof comprising a composition of the invention, packaged together with instructions for its use.

The present invention also provides methods for inducing and/or enhancing homing of a population of cells to a therapeutic target in a subject in need thereof, the method comprising: (a) providing to the population of cells a nucleic acid encoding a polypeptide, which enforces transient expression of a ligand that binds to a receptor at the therapeutic target; and (b) allowing the population of cells to express the polypeptide, wherein upon expression of the polypeptide homing of one or more cells in the population to a therapeutic target is induced and/or enhanced.

### BRIEF DESCRIPTION OF THE DRAWINGS

FIG. 1A-FIG. 1C show characterization of MSCs. FIG. 1A shows flow cytometry histograms of cell surface markers measured on a representative primary MSC line. FIG. 1B shows mean fluorescence intensity levels for the same markers as in panel A, displayed for all 7 primary MSC lines tested. Each MSC line was isolated from a different healthy donor. FIG. 1C shows photomicrographs of MSCs subjected to osteogenic differentiation conditions (bottom left panel), adipogenic differentiation conditions (bottom right panels), or MSC maintenance media (top panels). Cells were stained with Alizarin Red to detect calcified deposits, or Oil Red O to detect lipid deposits (scale bar=100μm).

FIG. 2 shows kinetics of sLe^{X} surface expression following intracellular or extracellular fucosylation of MSCs. Untreated MSCs, extracellularly fucosylated (FTVI-exo) MSCs, or intracellularly fucosylated (FUT6-mod) MSCs were harvested at 24-hour intervals, stained for sLe^{X} using HECA452 antibody, and analyzed by flow cytometry. MFI: Mean fluorescence intensity.

FIG. 3A-FIG. 3B show cell surface sLe^{X} expression levels induced by intracellular or extracellular fucosylation in multiple primary human MSC lines. FIG. 3A shows day 0 extracellularly fucosylated (FTVI-exo) MSCs and day 2-3 intracellularly fucosylated (FUT6-mod) MSCs show similar increase in surface sLe^{X} compared to untreated MSCs, as measured via flow cytometry analysis of HECA452 or csLex1 staining. FIG. 3B shows similar increase in surface sLe^{X} observed across multiple independent primary MSC lines (n=11 experiments; each color represents 1 of 5 primary MSC lines used. Statistical comparisons made using Student's T-test. n.s.=not significant (i.e. p>0.05). **** indicates p<0.0001.

FIG. 4A-FIG. 4D show assessment of MSC properties before and after intracellular or extracellular fucosylation. FIG. 4A shows percent viability of fucosylated MSCs measured by Trypan blue exclusion. Error bars=SEM. FIG. 4B shows cell surface marker expression for a primary MSC line before and after extracellular (FTVI-exo) or intracellular (FUT6-mod) fucosylation. FIG. 4C shows average (bar) and range (error bars) of mean fluorescence intensities of a panel of positive and negative markers for 2 primary MSC lines measured immediately after fucoslation (left panel) or when re-plated and cultured for one passage thereafter (i.e. 5-11 additional days) (right panel). FIG. 4D shows one primary MSC line was treated with FTVI exofucosylation or buffer alone, transfected with FUT6-modRNA or a control modRNA, or left untreated, followed by plating in triplicate and osteogenic differentiation was induced. Alizarin Red staining was measured to assess the overall amount of calcified deposits formed in each culture. Statistical comparisons were made using one-way ANOVA with Tukey's HSD test. n.s.=not significant (i.e. p>0.05); **=p<0.01.

FIG. 5A-FIG. 5B show a comparison of protein size and cellular localization of E-selectin ligand glycoproteins created by intracellular or extracellular fucosylation. FIG. 5A shows untreated MSCs, intracellularly fucosylated (FUT6-mod) MSCs, and extracellularly fucosylated (FTVI-exo) MSCs were lysed and Western blotted using mouse E-selectin-human Fc (E-Ig) chimera as a probe. FIG. 5A shows cellular localization of E-Ig reactive glycoproteins determined by treatment of intact intracellularly or extracellularly fucosylated MSCs with or without neuraminidase (NAse) prior to cell lysis and E-Ig Western blot. β-actin staining of same blots were performed as loading control.

FIG. 6A-FIG. 6B show that the about 85 kD E-selectin ligand in fucosylated MSCs is HCELL, an E-selectin binding CD44 glycoform. FIG. 6A shows E-selectin ligands from untreated, intracellularly fucosylated (FUT6-mod), and extracellularly fucosylated (FTVI-exo) MSC lysates were pulled down using E-Ig chimera, and Western blotted with CD44 antibody. FIG. 6B shows CD44 was immunoprecipitated from untreated, intracellularly fucosylated (FUT6-mod), and extracellularly fucosylated (FTVI-exo) MSC lysates, and Western blotted with the mAb HECA452, which recognizes sLe^{X}.

FIG. 7 shows an analysis of E-selectin ligand glycoproteins accessible to cell surface biotinylation. Untreated MSCs or intracellularly fucosylated (FUT6-mod) MSCs were incubated in-flask with amine-reactive biotinylation reagent, followed by extracellular fucosylation of a portion of the untreated MSCs (FTVI-exo). Untreated, FUT6-mod, and FTVI-exo cell lysates were separated into pulldown (biotinylated) and supernatant (non-biotinylated) fractions. Western blot was performed using E-selectin-Ig chimera and β-actin, as a loading control.

FIG. 8A-FIG. 8B show an analysis of E-selectin ligand mediated MSC-endothelial cell interactions under shear conditions using parallel plate flow chamber. (A) Both extracellular fucosylation (FTVI-exo) and intracellular fucosylation (FUT6-mod) enabled MSC capture/tethering/rolling under flow conditions on TNFα-activated human umbilical vein endothelial cells (HUVECs), but not on HUVECs pretreated with an anti-E-selectin function-blocking mAb. Error bars=SEM, n=4 independent experiments using 2 different primary MSC lines. (B) Extracellularly fucosylated and intracellularly fucosylated MSCs show similar rolling velocities on TNFα-stimulated HUVECs. Error bars=SEM, n=15 to 155 cell velocities analyzed per time point. Statistical comparisons made using Student's T-test. n.s.=not significant.

FIG. 9 shows efficacy of fucosylation confirmed in aliquots of DiD and Dil labeled MSC mixtures at time of xenotransplantation. FTVI exofucosylated (FTVI-exo) and buffer control MSCs, or FUT6-modRNA (FUT6-mod) and ndGFP control modRNA transfected MSCs, were labeled with Dil (blue) or DiD (green), mixed at 1:1 ratios, and injected into mice. Aliquots of each injected cell mixture were stained with sLe^{X} binding mAb HECA452 (red) and imaged on glass slides to confirm the efficacy of the FUT6-mod or FTVI-exo treatment, and to provide a precise starting ratio. Scale bar=100 μm.

FIG. 10A-FIG. 10C show in vivo imaging of calvarial bone marrow to measure relative osteotropism of xenotransplanted human MSCs. FIG. 10A shows three-dimensional reconstruction of mouse calvarium region after transplantation of DiD-(green) and Dil-(blue) stained MSCs. A portion of the bone is digitally removed to facilitate visualization of the bone marrow. Scale bar=100μm. FIG. 10B shows fucosylated human MSCs show increased osteotropism compared to control cells at 2 hours post-transplantation and FIG. 10C shows data from 24 hours post-transplantation, with intracellular fucosylation (FUT6-mod) yielding a stronger enhancement than extracellular fucosylation (FTVI-exo). Error bars=standard deviation. n=4 mouse pairs per comparison. Statistical comparisons were made using one-way ANOVA with Tukey's HSD test. *=p<0.05; **=p<0.01.

FIG. 11A-FIG. 11B show in vivo imaging of blood vessels to measure extravasation of xenotransplanted human MSCs into bone marrow parenchyma. FIG. 11A shows 2D merged image stack of calvarium region after Angiosense injection to visualize blood vessels (red) and homed Dil-(blue) and DiD-(green) stained MSCs. Scale bar=100μm. FIG. 11B shows intracellularly fucosylated (FUT6-mod) MSCs show significantly greater MSC extravasation into bone marrow parenchyma than do extracellularly fucosylated (FTVI-exo) MSCs when compared to control cells (baseline) at 24 hours post-transplantation. Error bars=standard deviation. n=4 mouse pairs per comparison. Statistical comparisons were made using one-way ANOVA with Tukey's HSD test. **=p<0.01.

### DETAILED DESCRIPTION OF THE INVENTION

In some embodiments, the present invention provides a method of enforcing expression of an E-selectin and/or L-selectin ligand on a surface of a cell, the method comprising the steps of: providing to the cell a nucleic acid encoding a glycosyltransferase, and culturing the cell under conditions sufficient to express the glycosyltransferase, wherein the expressed glycosyltransferase modifies a terminal sialylated lactosamine present on a glycoprotein of the cell to enforce expression the E-selectin and/or L-selectin ligand.

Glycosyltransferases are enzymes that catalyze the formation of the glycosidic linkage to form a glycoside. These enzymes utilize ‘activated’ sugar phosphates as glycosyl donors, and catalyze glycosyl group transfer to a nucleophilic group. The product of glycosyl transfer may be an O-, N-, S-, or C-glycoside; the glycoside may be part of a monosaccharide, oligosaccharide, or polysaccharide. The glycosyltransferases have been classified into more than 90 families. In some embodiments, the glycosyltransferase is an alpha 1,3-fucosyltransferase. Non-limiting examples of glycosyltransferases can be found, e.g., in C. Bretonet al.; Structures and mechanisms of glycosyltransferases, Glycobiology 2006; 16 (2): 29R-37R; D. Liang et al.; Glycosyltransferases: mechanisms and applications in natural product development, Chem. Soc. Rev., 2015, 44, 8350-8374; and Taniguchi et al; Handbook of Glycosyltransferases and Related Genes, Springer Science & Business Media, 2011. In some embodiments the cell is provided with nucleic acid encoding more than one glycosyltransferase. For example nucleic acids encoding two glycosyltransferases can be provided simultaneously or sequentially each adding a saccharide in an appropriate linkage to an extending core glycan structure. In some embodiments, the glycosyltransferase directs N-linked glycosylation. In other embodiments, the glycosyltransferase directs O-linked glycosylation. In some embodiments the alpha 1,3-fucosyltransferase is alpha 1,3-fucosyltransferase FTIII, FTIV, FTV, FTVI, FTVII, and combinations thereof.

In some embodiments the glycosyltransferase modifies the terminal sialylated lactosamine intracellularly.

In some embodiments, the present invention provides a method of enabling and/or increasing binding of a cell to E-selectin and/or L-selectin, the method comprising the steps of: providing to the cell a nucleic acid encoding an alpha 1,3-fucosyltransferase and culturing the cell under conditions sufficient for expression of the alpha 1,3-fucosyltransferase by the cell, wherein the alpha 1,3-fucosyltransferase modifies a glycan chain present on a glycoprotein to create an E-selectin and/or L-selectin ligand and thereby enable and/or increase the binding of the cell to E-selectin and/or L-selectin.

As used herein, “nucleic acid” or “oligonucleotide” or “polynucleotide” means at least two nucleotides covalently linked together. Many variants of a nucleic acid may be used for the same purpose as a given nucleic acid. Thus, a nucleic acid also encompasses substantially identical nucleic acids and complements thereof.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as disclosed in Krutzfeldt et al., Nature (Oct. 30, 2005), Soutschek et al., Nature 432:173-178 (2004), and U.S. Patent Application Publication No. 20050107325. Modified nucleotides and nucleic acids may also include locked nucleic acids (LNA), as disclosed in U.S. Patent Application Publication No. 20020115080. Additional modified nucleotides and nucleic acids are disclosed in U.S. Patent Application Publication No. 20050182005. Modifications of the ribose-phosphate backbone may be done for a variety of reasons, e.g., to increase the stability and half-life of such molecules in physiological environments, to enhance diffusion across cell membranes, etc. Mixtures of naturally occurring nucleic acids and analogs may be made; alternatively, mixtures of different nucleic acid analogs, and mixtures of naturally occurring nucleic acids and analogs may be made.

In some embodiments the cell is a mammalian cell. In some preferred aspects of these embodiments, the cell is a human cell.

In other embodiments the cell is a stem cell. In some preferred aspects of these embodiments, the stem cell is selected from the group consisting of embryonic stem cells, adult stem cells hematopoietic stem cells and induced pluripotent stem cells (iPSCs). In some preferred aspects of these embodiments, the adult stem cell is a mesenchymal stem cell.

As used herein, “providing a nucleic acid to a cell” and similar grammatical forms is intended to cover any conventional or to be discovered method of introducing a nucleotide sequence into a cell and expressing it. The expression may be long-term or transient and may be inducible or otherwise controlled using conventional methods known to those of skill in the art. In some embodiments the nucleic acid is provided to the cell by transfection. In other embodiments the nucleic acid is provided to the cell by transduction.

As used herein, “transfection” is a chemically mediated method of introducing a nucleic acid into a target cell. Non-limiting examples of transfection include lipid-based transfection and calcium phosphate based transfection. As used herein, “transduction” is a virally mediated method of introducing a nucleic acid into a target cell. Methods of transfection and transduction are known to those skilled in the art and can be selected to achieve effective delivery of a nucleic acid based on factors known to those skilled in the art such as cell type.

In some embodiments the nucleic acid is selected from the group consisting of a DNA, an RNA, a DNA/RNA hybrid, a cDNA, an mRNA, modified versions thereof, and combinations thereof. In preferred embodiments the nucleic acid is a modified RNA, in more preferred embodiments the modified RNA is modRNA.

As used herein a “modified RNA” includes base substitutions, backbone modifications, modifications to the 5′ or 3′ end, and combinations thereof.

As used herein “modRNA” is a modified RNA where cytidine and uridine are replaced with 5-methylcitidine and pseudouridine, respectively. A non-limiting example of a modRNA and how to make it is set forth in Example 1.

In some embodiments the alpha 1,3-fucosyltransferase is a human alpha 1,3-fucosyltransferase. In preferred embodiments the alpha 1,3-fucosyltransferase is human FTVI.

In some embodiments the alpha 1,3-fucosyltransferase fucosylates a glycoprotein selected from the group consisting of PSGL-1, CD43, CD44, and combinations thereof.

In other embodiments, the present invention provides a method of increasing homing and/or extravasation in a population of cells transplanted into a subject, the method comprising the steps of: providing to the population of cells a nucleic acid encoding an alpha 1,3-fucosyltransferase; culturing the population of cells under conditions sufficient for expression of the alpha 1,3-fucosyltransferase by one or more modified cells within the population, wherein the alpha 1,3-fucosyltransferase fucosylates a glycan chain present on a glycoprotein to create modified cells in which E-selectin and/or L-selectin ligand expression is enforced; and transplanting the population of cells into the subject, wherein the modified cells having enforced E-selectin and/or L-selectin ligand expression display increased homing and/or extravasation to therapeutically useful sites.

As used herein “enforcing expression of an E-selectin and/or L-selectin ligand” means to cause a glycan chain of a glycoprotein to be modified, e.g. by fucosylation, such that it is capable of functioning as a ligand for E-selectin and/or L-selectin. Enforcing expression of an E-selectin and/or L-selectin ligand can be accomplished, for example, by providing a glycosyltransferase, e.g. an alpha 1,3-fucosyltransferase, which can fucosylate a glycan chain of a glycoprotein present in or on the cell.

As used herein, a “subject” is a mammal, preferably, a human. In addition to humans, categories of mammals within the scope of the present invention include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

In some embodiments the population of cells is a population of mammalian cells. In some preferred aspects of these embodiments, the population of cells is a population of human cells.

In some embodiments the population of cells is a population of stem cells. In some preferred aspects of these embodiments, the population of stem cells is selected from the group consisting of embryonic stem cells, adult stem cells, hematopoietic stem cells and induced pluripotent stem cells (iPSCs). In some preferred aspects of these embodiments, the adult stem cells are mesenchymal stem cells.

“Transplanting” in the present invention includes all conventional and to be discovered methods of providing therapeutic compositions, e.g., a population of cells to an individual. The transplantation may be of the subject's own cells or from non-autologous donors. In some embodiments the step of transplanting occurs intravenously. In other embodiments the step of transplanting occurs near the site of desired extravasation.

In other embodiments, the present invention provides a method of producing modified cells for transplanting into a subject in need thereof, the method comprising the steps of: obtaining a population of cells to be modified; providing to the population of cells a nucleic acid encoding an alpha 1,3-fucosyltransferase; and culturing the population of cells under conditions sufficient for expression of the alpha 1,3-fucosyltransferase by one or more modified cells within the population, wherein the alpha 1,3-fucosyltransferase modifies a glycan chain present on a glycoprotein to create an E-selectin and/or L-selectin ligand.

The present invention also provides methods of producing modified stem cells for transplanting into a subject, the method comprising the steps of: obtaining a population of stem cells to be modified; providing to the population of stem cells a cDNA or modified RNA encoding an alpha 1,3-fucosyltransferase; and culturing the population of stem cells under conditions sufficient for expression of the alpha 1,3-fucosyltransferase by one or more modified cells within the population, wherein the expressed alpha 1,3-fucosyltransferase fucosylates CD44 present on or in the one or more modified cells.

In some additional embodiments the methods of the invention further comprise the step of carrying out extracellular fucosylation of CD44 expressed on the surface of the stem cells. As used herein “extracellular fucosylation” means providing an exogenous fucosyltransferase, e.g., FTIII, FTIV, FTV, FTVI, FTVII, or combinations thereof to the cells, e.g., stem cells as disclosed, e.g., in Sackstein et al. “Ex vivo glycan engineering of CD44 programs human multipotent mesenchymal stromal cell trafficking to bone” Nature Medicine. 2008;14:181-187 and Sackstein et al. “Glycosyltransferase-programmed stereosubstitution (GPS) to create HCELL: engineering a roadmap for cell migration” Immunol Rev. 2009;230:51-74.

The present invention also provides methods of treating or ameliorating the effects of a symptom, a disease or an injury in a subject in need thereof, the method comprising the steps of: obtaining a population of cells produced by any of the methods of the present invention; and transplanting an effective amount of the population of cells into the subject, wherein the transplanted cells extravasate to a site expressing E-selectin and/or L-selectin so as thereby to treat or ameliorate the effects of the symptom, disease or injury in the subject.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, the terms “ameliorate”, “ameliorating” and grammatical variations thereof mean to decrease the severity of the symptoms of a disease in a subject.

In the present invention, an “effective amount” or a “therapeutically effective amount” of an agent of the invention including pharmaceutical compositions containing same that are disclosed herein is an amount of such agent or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the duration of the treatment, the identity of any other agents being administered, the age, size, and species of mammal, e.g., human patient, and like factors well known in the arts of medicine and veterinary medicine. In general, a suitable amount of an agent or composition according to the invention will be that amount of the agent or composition, which is the lowest amount effective to produce the desired effect. The effective amount of an agent or composition of the present invention may be administered as two, three, four, five, six or more sub-doses, administered separately at appropriate intervals.

In some embodiments the disease is selected from the group consisting of an inflammatory disorder, an autoimmune disease, a degenerative disease, cardiovascular disease, ischemic disease, cancer, a genetic disease, a metabolic disorder and an idiopathic disorder.

In some embodiments the injury is selected from the group consisting of a physical injury, adverse drug effects, toxic injury, and an iatrogenic condition.

In some embodiments the subject is a mammal. In some preferred embodiments the mammal is selected from the group consisting of humans, primates, farm animals, and domestic animals. In some more preferred embodiments the mammal is human.

In some embodiments the transplanting occurs intravenously. In other embodiments the transplanting occurs near the site of desired extravasation. In some preferred embodiments the site of desired extravasation is the bone marrow. In other preferred embodiments the site of desired extravasation is the site of an injury or inflammation.

In other embodiments, the present invention provides a pharmaceutical composition comprising a population of cells produced by the methods of the invention and a pharmaceutically acceptable carrier.

In other embodiments, the present invention provides a kit for treating or ameliorating the effects of a symptom, a disease or an injury in a subject in need thereof comprising a composition of the invention, packaged together with instructions for its use.

The kits may also include suitable storage containers, e.g., ampules, vials, tubes, etc., for each pharmaceutical composition and other reagents, e.g., buffers, balanced salt solutions, etc., for use in administering the pharmaceutical compositions to subjects. The pharmaceutical compositions and other reagents may be present in the kits in any convenient form, such as, e.g., in a solution or in a powder form. The kits may further include instructions for use of the pharmaceutical compositions. The kits may further include a packaging container, optionally having one or more partitions for housing the pharmaceutical composition and other optional reagents.

The present invention also provides methods for inducing and/or enhancing homing of a population of cells to a therapeutic target in a subject in need thereof, the method comprising: (a) providing to the population of cells a nucleic acid encoding a polypeptide, which enforces transient expression of a ligand that binds to a receptor at the therapeutic target; and (b) allowing the population of cells to express the polypeptide, wherein upon expression of the polypeptide homing of one or more cells in the population to a therapeutic target is induced and/or enhanced.

In some embodiments, the population of cells is any medically relevant population, e.g., the population of cells may be selected from the group consisting of stem cells, tissue progenitor cells, antigen-specific T-cells, T-regulator cells, antigen-pulsed dendritic cells, NK cells, NKT cells, and leukocytes. In some embodiments the population of cells are T-lymphocytes. In some embodiments the population of cells are chimeric antigen receptor T-cells.

In some embodiments, the population of cells is culture-expanded prior to step (a).

In some embodiments, the therapeutic target may be any medically appropriate target, such as, e.g., a site of injury, inflammation, or a tumor.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

### EXAMPLES

Human mesenchymal stem cells (MSCs) hold great promise in cellular therapeutics for skeletal diseases but lack expression of E-selectin ligands that direct homing of blood-borne cells to bone marrow. Previously, we described a method to engineer E-selectin ligands on the MSC surface by exofucosylating cells with fucosyltransferase VI (FTVI) and its donor sugar, GDP-Fucose, enforcing transient surface expression of the potent E-selectin ligand HCELL with resultant enhanced osteotropism of intravenously administered cells. Here, we sought to determine whether E-selectin ligands created via FTVI-exofucosylation are distinct in identity and function to those created by FTVI expressed intracellularly. To this end, in the present Examples, we introduced synthetic modified mRNA encoding FTVI (FUT6-modRNA) into human MSCs. FTVI-exofucosylation extracellular fucosylation) and FUT6-modRNA transfection (i.e., intracellular fucosylation) produced similar peak increases in cell surface E-selectin ligand levels, and shear-based functional assays showed comparable increases in tethering/rolling on human endothelial cells expressing E-selectin. However, biochemical analyses revealed that intracellular fucosylation induced expression of both intracellular and cell surface E-selectin ligands and also induced a more sustained expression of E-selectin ligands compared to extracellular fucosylation. Notably, live imaging studies to assess homing of human MSC to mouse calvarium revealed more osteotropism following intravenous administration of intracellularly-fucosylated cells compared to extracellularly-fucosylated cells. This study represents the first direct analysis of E-selectin ligand expression programmed on human MSCs by FTVI-mediated intracellular versus extracellular fucosylation. The observed differential biologic effects of FTVI activity in these two contexts may yield new strategies for improving the efficacy of human MSCs in clinical applications.

### Example 1

### Materials and Methods

### Human Alpha 1,3 Fucosyltransferase Genes

Exemplary sequences of human proteins FUT3, FUT4, FUT5, FUT6 and FUT7 are shown below. Exemplary nucleic acid sequences encoding such fucosyltransferases for expression may encode the full length sequence (also shown below) or a truncated portion thereof which retains enzyme activity.

Human FUT3 cDNA sequence.

| SEQ ID NO: 1 | |
|---|---|
| 1 | aggaaacctg ccatggcctc ctggtgagct gtcctcatcc actgctcgct gcctctccag |
| 61 | atactctgac ccatggatcc cctgggtgca gccaagccac aatggccatg gcgccgctgt |
| 121 | ctggccgcac tgctatttca gctgctggtg gctgtgtgtt tcttctccta cctgcgtgtg |
| 181 | tcccgagacg atgccactgg atcccctagg gctcccagtg ggtcctcccg acaggacacc |
| 241 | actcccaccc gccccaccct cctgatcctg ctatggacat ggcctttcca catccctgtg |
| 301 | gctctgtccc gctgttcaga gatggtgccc ggcacagccg actgccacat cactgccgac |
| 361 | cgcaaggtgt acccacaggc agacacggtc atcgtgcacc actgggatat catgtccaac |
| 421 | cctaagtcac gcctcccacc ttccccgagg ccgcaggggc agcgctggat ctggttcaac |
| 481 | ttggagccac cccctaactg ccagcacctg gaagccctgg acagatactt caatctcacc |
| 541 | atgtcctacc gcagcgactc cgacatcttc acgccctacg gctggctgga gccgtggtcc |
| 601 | ggccagcctg cccacccacc gctcaacctc tcggccaaga ccgagctggt ggcctgggcg |
| 661 | gtgtccaact ggaagccgga ctcagccagg gtgcgctact accagagcct gcaggctcat |
| 721 | ctcaaggtgg acgtgtacgg acgctcccac aagcccctgc ccaaggggac catgatggag |
| 781 | acgctgtccc ggtacaagtt ctacctggcc ttcgagaact ccttgcaccc cgactacatc |
| 841 | accgagaagc tgtggaggaa cgccctggag gcctgggccg tgcccgtggt gctgggcccc |
| 901 | agcagaagca actacgagag gttcctgcca cccgacgcct tcatccacgt ggacgacttc |
| 961 | cagagcccca aggacctggc ccggtacctg caggagctgg acaaggacca cgcccgctac |
| 1021 | ctgagctact ttcgctggcg ggagacgctg cggcctcgct ccttcagctg ggcactggat |
| 1081 | ttctgcaagg cctgctggaa actgcagcag gaatccaggt accagacggt gcgcagcata |
| 1141 | gcggcttggt tcacctgaga ggccggcatg gtgcctgggc tgccgggaac ctcatctgcc |
| 1201 | tggggcctca cctgctggag tcctttgtgg ccaaccctct ctcttacctg ggacctcaca |
| 1261 | cgctgggctt cacggctgcc aggagcctct cccctccaga agacttgcct gctagggacc |
| 1321 | tcgcctgctg gggacctcgc ctgttgggga cctcacctgc tggggacctc acctgctggg |
| 1381 | gaccttggct gctggaggct gcacctactg aggatgtcgg cggtcgggga ctttacctgc |
| 1441 | tgggacctgc tcccagagac cttgccacac tgaatctcac ctgctgggga cctcaccctg |
| 1501 | gagggccctg ggccctgggg aactggctta cttggggccc cacccgggag tgatggttct |
| 1561 | ggctgatttg tttgtgatgt tgttagccgc ctgtgagggg tgcagagaga tcatcacggc |
| 1621 | acggtttcca gatgtaatac tgcaaggaaa aatgatgacg tgtctcctca ctctagaggg |
| 1681 | gttggtccca tgggttaaga gctcacccca ggttctcacc tcaggggtta agagctcaga |
| 1741 | gttcagacag gtccaagttc aagcccagga ccaccactta tagggtacag gtgggatcga |
| 1801 | ctgtaaatga ggacttctgg aacattccaa atattctggg gttgagggaa attgctgctg |
| 1861 | tctacaaaat gccaagggtg gacaggcgct gtggctcacg cctgtaattc cagcactttg |
| 1921 | ggaggctgag gtaggaggat tgattgaggc caagagttaa agaccagcct ggtcaatata |
| 1981 | gcaagaccac gtctctaaat aaaaaataat aggccggcca ggaaaaaaaa aaaaaaaaaa |
| 2041 | aaa |

Human FUT3 protein sequence.

SEQ ID NO: 2

```
                10 20 30 40
MDPLGAAKPQ WPWRRCLAAL LFQLLVAVCF FSYLRVSRDD
                50 60 70 80
ATGSPRAPSG SSRQDTTPTR PTLLILLWTW PFHIPVALSR
                90 100 110 120
CSEMVPGTAD CHITADRKVY PQADTVIVHH WDIMSNPKSR
              130 140 150 160
LPPSPRPQGQ RWIWFNLEPP PNCQHLEALD RYFNLTMSYR
              170 180 190 200
SDSDIFTPYG WLEPWSGOPA HPPLNLSAKT ELVAWAVSNW
              210 220 230 240
KPDSARVRYY QSLQAHLKVD VYGRSHKPLP KGTMMETLSR
              250 260 270 280
YKFYLAFENS LHPDYITEKL WRNALEAWAV PVVLGPSRSN
              290 300 310 270
YERFLPPDAF IHVDDFQSPK DLARYLQELD KDHARYLSYF
              330 340 350 360
RWRETLRPRS FSWALDFCKA CWKLQQESRY QTVRSIAAWY
T
```

Human FUT4 cDNA sequence.

| SEQ ID NO: 3 | |
|---|---|
| 1 | cgctcctcca cgcctgcgga cgcgtggcga gcggaggcag cgctgcctgt tcgcgccatg |
| 61 | ggggcaccgt ggggctcgcc gacggcggcg gcgggcgggc ggcgcgggtg gcgccgaggc |
| 121 | cgggggctgc catggaccgt ctgtgtgctg gcggccgccg gcttgacgtg tacggcgctg |
| 181 | atcacctacg cttgctgggg gcagctgccg ccgctgccct gggcgtcgcc aaccccgtcg |
| 241 | cgaccggtgg gcgtgctgct gtggtgggag cccttcgggg ggcgcgatag cgccccgagg |
| 301 | ccgccccctg actgccggct gcgcttcaac atcagcggct gccgcctgct caccgaccgc |
| 361 | gcgtcctacg gagaggctca ggccgtgctt ttccaccacc gcgacctcgt gaaggggccc |
| 421 | cccgactggc ccccgccctg gggcatccag gcgcacactg ccgaggaggt ggatctgcgc |
| 481 | gtgttggact acgaggaggc agcggcggcg gcagaagccc tggcgacctc cagccccagg |
| 541 | cccccgggcc agcgctgggt ttggatgaac ttcgagtcgc cctcgcactc cccggggctg |
| 601 | cgaagcctgg caagtaacct cttcaactgg acgctctcct accgggcgga ctcggacgtc |
| 661 | tttgtgcctt atggctacct ctaccccaga agccaccccg gcgacccgcc ctcaggcctg |
| 721 | gccccgccac tgtccaggaa acaggggctg gtggcatggg tggtgagcca ctgggacgag |
| 781 | cgccaggccc gggtccgcta ctaccaccaa ctgagccaac atgtgaccgt ggacgtgttc |
| 841 | ggccggggcg ggccggggca gccggtgccc gaaattgggc tcctgcacac agtggcccgc |
| 901 | tacaagttct acctggcttt cgagaactcg cagcacctgg attatatcac cgagaagctc |
| 961 | tggcgcaacg cgttgctcgc tggggcggtg ccggtggtgc tgggcccaga ccgtgccaac |
| 1021 | tacgagcgct ttgtgccccg cggcgccttc atccacgtgg acgacttccc aagtgcctcc |
| 1081 | tccctggcct cgtacctgct tttcctcgac cgcaaccccg cggtctatcg ccgctacttc |
| 1141 | cactggcgcc ggagctacgc tgtccacatc acctccttct gggacgagcc ttggtgccgg |
| 1201 | gtgtgccagg ctgtacagag ggctggggac cggcccaaga gcatacggaa cttggccagc |
| 1261 | tggttcgagc ggtgaagccg cgctcccctg gaagcgaccc aggggaggcc aagttgtcag |
| 1321 | ctttttgatc ctctactgtg catctccttg actgccgcat catgggagta agttcttcaa |
| 1381 | acacccattt ttgctctatg ggaaaaaaac gatttaccaa ttaatattac tcagcacaga |
| 1441 | gatgggggcc cggtttccat attttttgca cagctagcaa ttgggctccc tttgctgctg |
| 1501 | atgggcatca ttgtttaggg gtgaaggagg gggttcttcc tcaccttgta accagtgcag |
| 1561 | aaatgaaata gcttagcggc aagaagccgt tgaggcggtt tcctgaattt ccccatctgc |
| 1621 | cacaggccat atttgtggcc cgtgcagctt ccaaatctca tacacaactg ttcccgattc |
| 1681 | acgtttttct ggaccaaggt gaagcaaatt tgtggttgta gaaggagcct tgttggtgga |
| 1741 | gagtggaagg actgtggctg caggtgggac tttgttgttt ggattcctca cagccttggc |
| 1801 | tcctgagaaa ggtgaggagg gcagtccaag aggggccgct gacttctttc acaagtacta |
| 1861 | tctgttcccc tgtcctgtga atggaagcaa agtgctggat tgtccttgga ggaaacttaa |
| 1921 | gatgaataca tgcgtgtacc tcactttaca taagaaatgt attcctgaaa agctgcattt |
| 1981 | aaatcaagtc ccaaattcat tgacttaggg gagttcagta tttaatgaaa ccctatggag |
| 2041 | aatttatccc tttacaatgt gaatagtcat ctcctaattt gtttcttctg tctttatgtt |
| 2101 | tttctataac ctggattttt taaatcatat taaaattaca gatgtgaaaa taaaaaaaa |

Human FUT4 protein sequence.

SEQ ID NO: 4

```
                10 20 30 40
MRRLWGAARK PSGAGWEKEW AEAPQEAPGA WSGRLGPGRS
                50 60 70 80
GRKGRAVPGW ASWPAHLALA ARPARHLGGA GQGPRPLHSG
                90 100 110 120
TAPFHSRASG ERQRRLEPQL QHESRCRSST PADAWRAEAA
              130 140 150 160
LPVRAMGAPW GSPTAAAGGR RGWRRGRGLP WTVCVLAAAG
              170 180 190 200
LTCTALITYA CWGQLPPLPW ASPTPSRPVG VLLWWEPFGG
              210 220 230 240
RDSAPRPPPD CRLRFNISGC RLLTDRASYG EAQAVLFHHR
              250 260 270 280
DLVKGPPDWP PPWGIQAHTA EEVDLRVLDY EEAAAAAEAL
              290 300 310 320
ATSSPRPPGQ RWVWMNFESP SHSPGLRSLA SNLFNWTLSY
              330 340 350 360
RADSDVFVPY GYLYPRSHPG DPPSGLAPPL SRKQGLVAWV
              370 380 390 400
VSHWDERQAR VRYYHQLSQH VTVDVFGRGG PGQPVPEIGL
              410 420 430 440
LHTVARYKFY LAFENSQHLD YITEKLWRNA LLAGAVPVVL
              450 460 470 480
GPDRANYERF VPRGAFIHVD DFPSASSLAS YLLFLDRNPA
              490 500 510 520
VYRRYFHWRR SYAVHITSFW DEPWCRVCQA VQRAGDRPKS
              530
IRNLASWFER
```

Human FUT5 cDNA sequence.

| SEQ ID NO: 5 | |
|---|---|
| 1 | tttatgacaa gctgtgtcat aaattataac agcttctctc aggacactgt ggccaggaag |
| 61 | tgggtgatct tccttaatga ccctcactcc tctctcctct cttcccagct actctgaccc |
| 121 | atggatcccc tgggcccagc caagccacag tggctgtggc gccgctgtct ggccgggctg |
| 181 | ctgtttcagc tgctggtggc tgtgtgtttc ttctcctacc tgcgtgtgtc ccgagacgat |
| 241 | gccactggat cccctaggcc agggcttatg gcagtggaac ctgtcaccgg ggctcccaat |
| 301 | gggtcccgct gccaggacag catggcgacc cctgcccacc ccaccctact gatcctgctg |
| 361 | tggacgtggc cttttaacac acccgtggct ctgccccgct gctcagagat ggtgcccggc |
| 421 | gcggccgact gcaacatcac tgccgactcc agtgtgtacc cacaggcaga cgcggtcatc |
| 481 | gtgcaccact gggatatcat gtacaacccc agtgccaacc tcccgccccc caccaggccg |
| 541 | caggggcagc gctggatctg gttcagcatg gagtccccca gcaactgccg gcacctggaa |
| 601 | gccctggacg gatacttcaa tctcaccatg tcctaccgca gcgactccga catcttcacg |
| 661 | ccctacggct ggctggagcc gtggtccggc cagcctgccc acccaccgct caacctctcg |
| 721 | gccaagaccg agctggtggc ctgggcggtg tccaactgga agccggactc ggccagggtg |
| 781 | cgctactacc agagcctgca ggctcatctc aaggtggacg tgtacggacg ctcccacaag |
| 841 | cccctgccca aggggaccat gatggagacg ctgtcccggt acaagttcta tctggccttc |
| 901 | gagaactcct tgcaccccga ctacatcacc gagaagctgt ggaggaacgc cctggaggcc |
| 961 | tgggccgtgc ccgtggtgct gggccccagc agaagcaact acgagaggtt cctgccgccc |
| 1021 | gacgccttca tccacgtgga tgacttccag agccccaagg acctggcccg gtacctgcag |
| 1081 | gagctggaca aggaccacgc ccgctacctg agctactttc gctggcggga gacgctgcgg |
| 1141 | cctcgctcct tcagctgggc actggctttc tgcaaggcct gctggaagct gcagcaggaa |
| 1201 | tccaggtacc agacggtgcg cagcatagcg gcttggttca cctgagaggc cggcatgggg |
| 1261 | cctgggctgc cagggacctc actttcccag ggcctcacct acctagggtc |

Human FUT5 protein sequence.

SEQ ID NO: 6

```
                10 20 30 40
MDPLGPAKPQ WLWRRCLAGL LFQLLVAVCF FSYLRVSRDD
                50 60 70 80
ATGSPRPGLM AVEPVTGAPN GSRCQDSMAT PAHPTLLILL
                90 100 110 120
WTWPFNTPVA LPRCSEMVPG AADCNITADS SVYPQADAVI
              130 140 150 160
VHHWDIMYNP SANLPPPTRP QGQRWIWFSM ESPSNCRHLE
              170 180 190 200
ALDGYFNITM SYRSDSDIFT PYGWLEPWSG QPAHPPLNLS
              210 220 230 240
AKTELVAWAV SNWKPDSARV RYYQSLQAHL KVDVYGRSHK
              250 260 270 280
PLPKGTMMET LSRYKFYLAF ENSLHPDYIT EKLWRNALEA
              290 300 310 320
WAVPVVLGPS RSNYERFLPP DAFIHVDDFQ SPKDLARYLQ
              330 340 350 360
ELDKDHARYL SYFRWRETLR PRSFSWALAF CKACWKLQQE
              370
SRYQTVRSIA AWFT
```

Human FUT6 cDNA sequence.

| SEQ ID NO: 7 | |
|---|---|
| 1 | cagatactct gacccatgga tcccctgggc ccggccaagc cacagtggtc gtggcgctgc |
| 61 | tgtctgacca cgctgctgtt tcagctgctg atggctgtgt gtttcttctc ctatctgcgt |
| 121 | gtgtctcaag acgatcccac tgtgtaccct aatgggtccc gcttcccaga cagcacaggg |
| 181 | acccccgccc actccatccc cctgatcctg ctgtggacgt ggccttttaa caaacccata |
| 241 | gctctgcccc gctgctcaga gatggtgcct ggcacggctg actgcaacat cactgccgac |
| 301 | cgcaaggtgt atccacaggc agacgcggtc atcgtgcacc accgagaggt catgtacaac |
| 361 | cccagtgccc agctcccacg ctccccgagg cggcaggggc agcgatggat ctggttcagc |
| 421 | atggagtccc caagccactg ctggcagctg aaagccatgg acggatactt caatctcacc |
| 481 | atgtcctacc gcagcgactc cgacatcttc acgccctacg gctggctgga gccgtggtcc |
| 541 | ggccagcctg cccacccacc gctcaacctc tcggccaaga ccgagctggt ggcctgggca |
| 601 | gtgtccaact gggggccaaa ctccgccagg gtgcgctact accagagcct gcaggcccat |
| 661 | ctcaaggtgg acgtgtacgg acgctcccac aagcccctgc cccagggaac catgatggag |
| 721 | acgctgtccc ggtacaagtt ctatctggcc ttcgagaact ccttgcaccc cgactacatc |
| 781 | accgagaagc tgtggaggaa cgccctggag gcctgggccg tgcccgtggt gctgggcccc |
| 841 | agcagaagca actacgagag gttcctgccg cccgacgcct tcatccacgt ggacgacttc |
| 901 | cagagcccca aggacctggc ccggtacctg caggagctgg acaaggacca cgcccgctac |
| 961 | ctgagctact ttcgctggcg ggagacgctg cggcctcgct ccttcagctg ggcactcgct |
| 1021 | ttctgcaagg cctgctggaa actgcaggag gaatccaggt accagacacg cggcatagcg |
| 1081 | gcttggttca cctgagaggc ccggcatggg gcctgggctg ccaggg |

Human FUT6 protein sequence.

SEQ ID NO: 8

```
                10 20 30 40
MDPLGPAKPQ WSWRCCLTTL LFQLLMAVCF FSYLRVSQDD
                50 60 70 80
PTVYPNGSRF PDSTGTPAHS IPLILLWTWP FNKPIALPRC
                90 100 110 120
SEMVPGTADC NITADRKVYP QADAVIVHHR EVMYNPSAQL
              130 140 150 160
PRSPRRQGQR WIWFSMESPS HCWQLKAMDG YFNLTMSYRS
              170 180 190 200
DSDIFTPYGW LEPWSGQPAH PPLNLSAKTE LVAWAVSNWG
              210 220 230 240
PNSARVRYYQ SLQAHLKVDV YGRSHKPLPQ GTMMETLSRY
              250 260 270 280
KFYLAFENSL HPDYITEKLW RNALEAWAVP VVLGPSRSNY
              290 300 310 320
ERFLPPDAFI HVDDFQSPKD LARYLQELDK DHARYISYFR
              330 340 350
WRETLRPRSF SWALAFCKAC WKLQEESRYQ TRGIAAWFT
```

Human FUT7 cDNA sequence.

| SEQ ID NO: 9 | |
|---|---|
| 1 | aaggagcaca gttccaggcg gggctgagct agggcgtagc tgtgatttca ggggcacctc |
| 61 | tggcggctgc cgtgatttga gaatctcggg tctcttggct gactgatcct gggagactgt |
| 121 | ggatgaataa tgctgggcac ggccccaccc ggaggctgcg aggcttgggg gtcctggccg |
| 181 | gggtggctct gctcgctgcc ctctggctcc tgtggctgct ggggtcagcc cctcggggta |
| 241 | ccccggcacc ccagcccacg atcaccatcc ttgtctggca ctggcccttc actgaccagc |
| 301 | ccccagagct gcccagcgac acctgcaccc gctacggcat cgcccgctgc cacctgagtg |
| 361 | ccaaccgaag cctgctggcc agcgccgacg ccgtggtctt ccaccaccgc gagctgcaga |
| 421 | cccggcggtc ccacctgccc ctggcccagc ggccgcgagg gcagccctgg gtgtgggcct |
| 481 | ccatggagtc tcctagccac acccacggcc tcagccacct ccgaggcatc ttcaactggg |
| 541 | tgctgagcta ccggcgcgac tcggacatct ttgtgcccta tggccgcctg gagccccact |
| 601 | gggggccctc gccaccgctg ccagccaaga gcagggtggc cgcctgggtg gtcagcaact |
| 661 | tccaggagcg gcagctgcgt gccaggctgt accggcagct ggcgcctcat ctgcgggtgg |
| 721 | atgtctttgg ccgtgccaat ggacggccac tgtgcgccag ctgcctggtg cccaccgtgg |
| 781 | cccagtaccg cttctacctg tcctttgaga actctcagca ccgcgactac attacggaga |
| 841 | aattctggcg caacgcactg gtggctggca ctgtgccagt ggtgctgggg cccccacggg |
| 901 | ccacctatga ggccttcgtg ccggctgacg ccttcgtgca tgtggatgac tttggctcag |
| 961 | cccgagagct ggcggctttc ctcactggca tgaatgagag ccgataccaa cgcttctttg |
| 1021 | cctggcgtga caggctccgc gtgcgactgt tcaccgactg gcgggaacgt ttctgtgcca |
| 1081 | tctgtgaccg ctacccacac ctaccccgca gccaagtcta tgaggacctt gagggttggt |
| 1141 | ttcaggcctg agatccgctg gccgggggag gtgggtgtgg gtggaagggc tgggtgtcga |
| 1201 | aatcaaacca ccaggcatcc ggcccttacc ggcaagcagc gggctaacgg gaggctgggc |
| 1261 | acagaggtca ggaagcaggg gtggggggtg caggtgggca ctggagcatg cagaggaggt |
| 1321 | gagagtggga gggaggtaac gggtgcctgc tgcggcagac gggaggggaa aggctgccga |
| 1381 | ggaccctccc caccctgaac aaatcttggg tgggtgaagg cctggctgga agagggtgaa |
| 1441 | aggcagggcc cttggggctg gggggcaccc cagcctgaag tttgtggggg ccaaacctgg |
| 1501 | gaccccgagc ttcctcggta gcagaggccc tgtggtcccc gagacacagg cacgggtccc |
| 1561 | tgccacgtcc atagttctga ggtccctgtg tgtaggctgg ggcggggccc aggagaccac |
| 1621 | ggggagcaaa ccagcttgtt ctgggctcag ggagggaggg cggtggacaa taaacgtctg |
| 1681 | agcagtgaaa aaaaaaaaaa a |

Human FUT7 protein sequence.

SEQ ID NO: 10

```
                10 20 30 40
MNNAGHGPTR RLRGLGVLAG VALLAALWLL WLLGSAPRGT
                50 60 70 80
PAPQPTITIL VWHWPFTDQP PELPSDTCTR YGIARCHLSA
                90 100 110 120
NRSLLASADA VVFHHRELQT RRSHLPLAQR PRGQPWVWAS
              130 140 150 160
MESPSHTHGL SHLRGIFNWV LSYRRDSDIF VPYGRLEPHW
              170 180 190 200
GPSPPLPARS RVAAWVVSNF QERQLRARLY RQLAPHLRVD
              210 220 230 240
VFGRANGRPL CASCLVPTVA QYRFYLSFEN SQHRDYITEK
              250 260 270 280
FWRNALVAGT VPVVLGPPRA TYEAFVPADA FVHVDDFGSA
              290 300 310 320
RELAAFLTGM NESRYQRFFA WRDRLRVRLF TDWRERFCAI
              330 340
CDRYPHLPRS QVYEDLEGWF QA
```

### Isolation and Culture of Human Mesenchymal Stem Cells

Human cells were obtained and used in accordance with the procedures approved by the Human Experimentation and Ethics Committees of Partners Cancer Care Institutions (Massachusetts General Hospital, Brigham and Women's Hospital, and Dana-Farber Cancer Institute). Discarded bone marrow filter sets were obtained from normal human donors. Bone marrow cells were flushed from the filter set using PBS plus 10 U/ml heparin (Hospira). The mononuclear fraction was isolated using density gradient media (Ficoll-Histopaque 1.077, Sigma-Aldrich) and suspended at 2-5×10⁶ cells/ml in MSC medium (DMEM 1 g/L glucose, 10% FBS from selected lots, 100 U/ml penicillin, 100 U/ml streptomycin). 20 m1 of cell suspension was seeded into T-175 tissue culture flasks and incubated at 37° C., 5% CO2, >95% humidity. 24 hours later, non-adherent cells were removed, the flask was rinsed with PBS, and fresh MSC medium was added. Subsequently, MSC media was exchanged twice per week. By 1-2 weeks, clusters of adherent MSCs were observed. When confluence approached 80%, cells were harvested and diluted 3- to 5-fold in MSC media and plated into new flasks. To harvest, MSCs were rinsed twice with PBS, and lifted with 0.05% trypsin and 0.5 mM EDTA. After centrifugation, the cell pellet was resuspended in MSC medium for passaging or washed with PBS for experimental use.

### MSC Characterization and Differentation

MSCs were characterized by FACS staining for a panel of markers, including CD29, CD31, CD34, CD45, CD73, CD90, CD105, CD106, and CD166. Cell viability was measured using Trypan Blue exclusion. To induce osteogenic differentiation, cells were cultured in the presence of MSC media plus 10 nM dexamethasone, 10 mM glycerophosphate, and 50 μg/ml L-ascorbate-2-phosphate. After 4 days, the L-ascorbate-2-phosphate was removed, and the media was changed every 3-4 days for a total of 14 days. To induce adipogenic differentiation, cells were cultured in DMEM with 3 ug/L glucose, 3% FBS, 1 μM dexamethasone, 500 μM methylisobutylmethylxanthine (IBMX), 33 μM biotin, 5 μM rosiglitazone, 100 nM insulin, and 17 μM pantothenate. After 4 days, the IBMX and rosiglitazone was removed, and the media was changed every 3-4 days for a total of 14 days. As negative control, MSCs were maintained in MSC media, changing every 3-4 days for a total of 14 days. To visualize calcified deposits indicative of osteogenic differentiation, cells were stained with 2% Alizarin Red. After photomicrographs were taken, the cells were destained using 10% cetylpyridinium chrloride monohydrate and the stained eluates were measured using a spectrophotometer at 595 nm. To visualize lipid deposits indicative of adipogenic differentiation, cells were stained with 0.3% Oil Red O, and micrographs were taken.

Modified mRNA Synthesis

Modified mRNA (modRNA) was synthesized as described previously [Mandal 2013]. Briefly, cDNA encoding human Fucosyltransferase 6 (FUT6) was sub-cloned into a vector containing T7 promoter, 5′ UTR and 3′ UTR. PCR reactions were performed to generate template for in vitro transcription with HiFi Hotstart (KAPA Biosystems). 1.6 μg of purified PCR product including FUT6 ORF and 5′ and 3′ UTR was used as template for RNA synthesis with MEGAscript T7 kit (Ambion). 3′-0-Me-m7G(5′)ppp(5′)G ARCA cap analog (New England Biolabs), adenosine triphosphate and guanosine triphosphate (USB), 5-methylcytidine triphosphate and pseudouridine triphosphate (TriLink Biotechnologies) were used for in vitro transcription reaction. modRNA product was purified using MEGAclear spin columns (Ambion), and aliquots were stored frozen for future use. Nuclear destabilized EGFP (ndGFP) modRNA was similarly prepared as a negative control.

modRNA Transfection

modRNA transfections were carried out with Stemfect (Stemgent) as per the manufacturer's instructions. Tubes were prepared with 1 μg of modRNA in 60 μl of buffer and 2 μl of reagent in 60 μl of buffer, then the two complexes were mixed together and incubated for 15 minutes at room temperature. The mixture was added to 1×10⁶ MSCs in 2 m1 of MSC medium. Subsequent to modRNA transfection, the B18R interferon inhibitor (eBioscience) was used as a media supplement at 200 ng/ml.

### FTVI Production and Specific Activity Measurement

Recombinant FTVI enzyme was produced in CHO cells by established techniques [Borsig 1998], using cDNA encoding amino acids 35-359 of the FTVI protein sequence (SEQ ID NO:8); this sequence omits the cytoplasmic and transmembrane regions of FTVI, and encompasses the entire stem and catalytic domain of the enzyme. The specific activity of the purified enzyme was determined using the Glycosyltransferase Activity Kit (R&D Systems), as per the manufacturer's instructions. Briefly, 0.1 μg of recombinant FTVI, 1 μL of ENTPD3/CD39L3 phosphatase, 15 nmol of N-acetyl-D-lactosamine (V-labs Inc), and 4 nmol of GDP-Fucose (Sigma-Aldrich) were mixed in 50 μL reaction buffer (25 mM Tris, 10 mM CaCl2 and 10 mM MnCl2, pH 7.5) and incubated in a 96-well plate at 37oC for 20 minutes. A second reaction that contained the same components except the recombinant FTVI was performed as a negative control. Reactions were terminated by the addition of 30 μL of Malachite Green Reagent A and 100 μL of water to each well. Color was developed by the addition of 30 μL of Malachite Green Reagent B to each well followed by gentle mixing and incubation at room temperature for 20 minutes. The plate was read at 620 nm using a multi-well plate reader. Phosphate standards were used to generate a calibration curve, and the specific activity of the FTVI enzyme was determined to be 60 pmol/min/μg.

### FTVI Exofucosylation

MSCs were harvested, washed twice with PBS, and resuspended at 2×10⁷ cells/ml in FTVI reaction buffer, containing 20 mM HEPES (Gibco), 0.1% human serum albumin (Sigma), 1 mM GDP-fucose (Carbosynth), and 60 μg/ml purified FTVI enzyme in Hank's Balanced Salt Solution (HBSS). Cells were incubated at 37° C. for 1 hour. For some experiments, “buffer only” controls were performed in an identical fashion but excluding the FTVI enzyme and GDP-fucose from the reaction. After the reaction, the cells were washed 2× with PBS and used immediately for downstream experiments.

### Flow Cytometry

2.5 μl HECA-FITC (Biolegend) or CsLex1-FITC (eBiosciences) were added to individual wells of 96-well plates. MSCs were harvested and suspended at 1×10⁶/ml in PBS plus 2% FBS, and 50 μl of cell suspension was added to each well. After 30 minutes incubation at 4° C., the plate was washed with 200 μl PBS per well and resuspended in 200 μl PBS. Fluorescence intensity was determined using a Cytomics FC 500 MPL flow cytometer (Beckman Coulter).

Time Course of Enforced sLe^{X} Expression Following FUT6-modRNA Transfection and FTVI Exofucosylation

MSCs were FUT6-modRNA transfected, FTVI exofucosylated, or left untreated, and an aliquot was removed for flow cytometric analysis for expression of sLe^{X} using mAb HECA452. Remaining cells were passaged into T-25 flasks (6 flasks per group). At 24 hour intervals, one flask from each group was harvested and flow cytometry was performed using HECA452. A time course of cell surface sLe^{X} expression was obtained by comparing the mean fluorescence intensity of HECA452 staining on each sample from day to day.

### Cell Surface Neuraminidase Treatment and Western Blot Analysis

Untreated, FUT6-modRNA transfected MSCs (day 3), and exofucosylated MSCs (day 0) MSCs were suspended at 10⁷ cells/ml in HBSS+0.1% BSA and incubated with or without 0.1 U/ml of Arthrobacter ureafasiens neuraminidase (Sigma) for 45 minutes at 37° C. MSCs were then washed, counted, pelleted and frozen at −80° C. Prior to use, lysates were prepared by adding 30 μl of twice reducing SDS-Sample Buffer per 10⁵ cells and boiling for 10 minutes. The samples were then separated on 7.5% Criterion Tris-HSC SDS-PAGE gels and transferred to PVDF membrane. Membranes were blocked with 5% milk and then stained consecutively with mouse E-selectin human-Ig chimera (E-Ig, R&D Systems), rat anti-mouse E-selectin (clone 10E9.6, BD Biosciences), and goat anti-rat IgG conjugated to horseradish peroxidase (HRP, Southern Biotech). All staining and washes were performed in Tris-buffered saline plus 0.1% Tween®20 plus 2 mM CaCl2. Blots were visualized with chemiluminescence using Lumi-Light Western Blotting Substrate (Roche) as per the manufacturer's instructions. To confirm equal loading, membranes were subsequently stained with rabbit anti-human beta-actin (ProSci) followed by goat anti-rabbit IgG-HRP (SouthernBiotech), and visualized with chemiluminescence as described.

### Immunoprecipitation and E-Selectin (E-Ig) Pulldown of HCELL

MSCs were FUT6-modRNA transfected, FTVI exofucosylated, or untreated (control), and lysates were prepared in 2% NP40, 150 mM NaCl, 50 mM Tris-HCl (pH7.4), 20 μg/mL PMSF, and 1× protease inhibitor cocktail (Roche). Cell lysates were precleared with protein G-agarose beads (Invitrogen). For CD44 immunoprecipitation, lysates were incubated with a cocktail of mouse anti-human CD44 monoclonal antibodies consisting of 2C5 (R&D Systems), F10-44.2 (Southern Biotech), 515 and G44-26 (both from BD Biosciences). For E-selectin pulldown, lysates were incubated with mouse E-Ig in the presence of 2 mM CaCl2. CD44 immunoprecipitates and E-Ig pulldowns were collected with protein G-agarose beads and eluted via boiling in 1.5× reducing SDS-Sample Buffer, run on an SDS-PAGE gel, and Western Blotted with anti-CD44 antibodies 2C5, G44-26, and F10-44.2, or the anti-sLe^{X} antibody HECA452.

### Cell Surface Protein Isolation

MSCs were biotinylated in-flask and cell surface proteins were isolated using the Pierce Cell Surface Protein Isolation Kit (Thermo Scientific), according to the manufacturer's instructions. Briefly, untreated MSCs or FUT6-modRNA transfected MSCs plated 3 days prior were rinsed with PBS, and 10 ml of amine-reactive EZ-Link Sulfo-NHS-SS-Biotin reagent was added to each flask. Flasks were gently agitated for 30 minutes at 4° C., and the reaction was quenched with lysine. Cells were harvested, and a portion of the untreated MSCs were exofucosylated with FTVI. After the exofucosylation reaction, cells were washed and lysed. Biotinylated cell surface proteins were isolated using the NeutrAvidin Agarose beads and the spin columns provided in the kit. The flow-through was collected as the non-biotinylated fraction, and the bound proteins were eluted and collected as the biotinylated (cell surface) fraction. These fractions were run on a gel and Western Blot was performed for E-Ig chimera and beta-actin as described.

### Parallel Plate Flow Chamber Studies

Parallel plate flow experiments were performed using a Bioflux-200 system and 48-well low-shear microfluidic plates (Fluxion Biosciences). Microfluidic chambers were coated with 250 μg/ml fibronectin (BD Biosciences) and seeded with human umbilical vein endothelial cells (HUVECs, Lonza), then cultured in endothelial growth media prepared from the EGM-2 BulletKit (EGM-2 media, Lonza) until confluent monolayers were formed. Four hours prior to assay, HUVECs were activated with 40 ng/ml rhTNFα (R&D Systems) to induce E-selectin expression. FUT6-modRNA transfected MSCs, FTVI exofucosylated MSCs, or untreated MSCs were suspended at 1.0-1.5×10⁶/ml in EGM-2 media and infused initially at a flow rate representing shear stress of 0.5 dynes/cm2, increasing at 1-minute intervals to 1, 2, 4, 8, and 16 dynes/cm2. The number of rolling cells captured per field was counted for two separate 10-second intervals at each flow rate, and averaged. Cell counts were corrected for starting cell number by visually determining the total number of cells visible per field in the initial infusate at 0.5 dynes/cm2, and expressing the captured cell numbers as a proportion of the starting cell number normalized to the number of cells at 1.0×10⁶ cells/ml. Data is thus presented as the number of rolling cells captured per mm2, normalized to 1×10⁶ cells/ml infusate. To determine the specificity of binding of the fucosylated cells, negative controls were performed using HUVECs not activated with TNFα, and also with activated HUVECs blocked with anti-CD62E (E-selectin) antibody (clone 68-5H11, BD Pharmingen). The blocking antibody was suspended at 20 μg/ml in EGM-2 media, infused onto the HUVECs and incubated for 20 minutes prior to washing and infusing the fucosylated MSCs. Rolling velocities were calculated by measuring the distance travelled in each 10 second interval for all rolling cells, converting to velocities measured in μm/second, and reporting the average rolling velocity for all rolling cells at each shear stress.

### Vital Dye Staining and Intravenous Infusion of Human MSC Into Mice

MSCs were harvested, transfected with FUT6-modRNA or ndGFP modRNA, and plated into T-175 flasks with B18R. Untreated MSCs were passaged at the same time. 2 days later, the untreated MSCs were harvested and split into FTVI-exofucosylation or “buffer only” control groups. FUT6 and ndGFP transfected MSCs were harvested directly. Aliquots of all samples were removed for flow cytometry analysis of HECA452. MSCs from each of the four treatments were split in two, suspended at 1×10⁶ cells/ml in PBS+0.1% BSA and stained with 10 μM Vybrant® DiD or Vybrant® Dil dyes (Molecular Probes) for 20 minutes at 37° C. Cells were washed twice, and 1:1 reciprocal mixtures (FUT6-modRNA transfected MSCs mixed 1:1 with ndGFP control transfected MSCs, and FTVI-exofucosylated MSCs mixed 1:1 with buffer control treated MSCs) were prepared. Pairs of immunocompetent BL/6 mice were retro-orbitally injected with each cell combination, with the membrane dye combination swapped between the mice in each pair. Subsequently, 2 nmol of Angiosense 750 (PerkinElmer) was injected per mouse to enable simultaneous visualization of blood vessels. Aliquots of the cell mixtures injected into each mouse were stained with HECA452-FITC and imaged on a glass slide to confirm the efficacy of the FUT6-mod or FTVI-exo treatment. A minimum of 20 such images (average 450 cells) were counted to provide a precise starting ratio of DiD and Dil labeled MSCs for each mouse. In cases where the starting ratio was different from 1:1, a correction factor was calculated and the homing ratios obtained from the in vivo images were adjusted accordingly.

In Vivo Confocal and 2-photon Fluorescence Microscopy

MSC homing to the in vivo calvarial bone marrow was imaged using a custom-built video-rate laser-scanning microscope designed for live animal imaging under isoflurane anesthesia. Scalp hair was shaved, and a skin flap was surgically opened, exposing the calvarium. The calvarial region was wetted with saline and positioned directly under a 60× 1.0NA water immersion objective lens (Olympus, Center Valley, Pa.). Image stacks were acquired at 30 frames per second, with frame averaging to enhance the signal-to-noise ratio. Dil-labeled MSCs, DiD-labeled MSCs, and Angiosense 750-labeled vasculature were imaged using a confocal detection scheme. Second harmonic generation of bone collagen was performed using 840 nm light from a femtosecond pulsed Maitai laser (Coherent, Inc., Santa Clara, Calif.). Cells could be detected to a depth of approximately 200 μm in the tissue. Imaging was performed at about 2 hours and about 24 hours post-transplant. Between imaging sessions, the scalp flap was stitched closed and the mouse was allowed to recover. Studies were in accordance with U.S. National Institutes of Health guidelines for care and use of animals under approval of the Institutional Animal Care and Use Committees of Massachusetts General Hospital.

### In Vivo Image Analysis

Calvarial images were collected and quantified as 3-dimensional stacks [Mortensen 2013]. For quantification, the numbers of DiD and Dil cells in 20 representative imaging locations across the bone marrow of the calvarium were manually counted for each mouse. Analysis was performed blinded, with counted events corresponding to a minimum diameter of about 10 μm to eliminate debris from analysis, and excluding autofluorescent events with signal in both DiD and Dil channels (those events with the intensity of the primary channel less than about 2× the intensity of the other channel). Extravasated cells were defined as those that were completely discrete from the Angiosense labeled vessels (i.e. no part of the cell was overlapping with any part of any vessel). The ratios of DiD to Dil stained cells counted in each mouse were calculated and compared within each mouse pair, with equivalent homing assigned a baseline ratio of 1. Fold change in homing of the treated MSCs compared to control MSCs was thus calculated for each pair of mice to provide a relative measurement of homing efficacy. 8 mice (4 mouse pairs) representing 4 different primary MSC lines were imaged per treatment.

### Example 2

### MSC Characterization

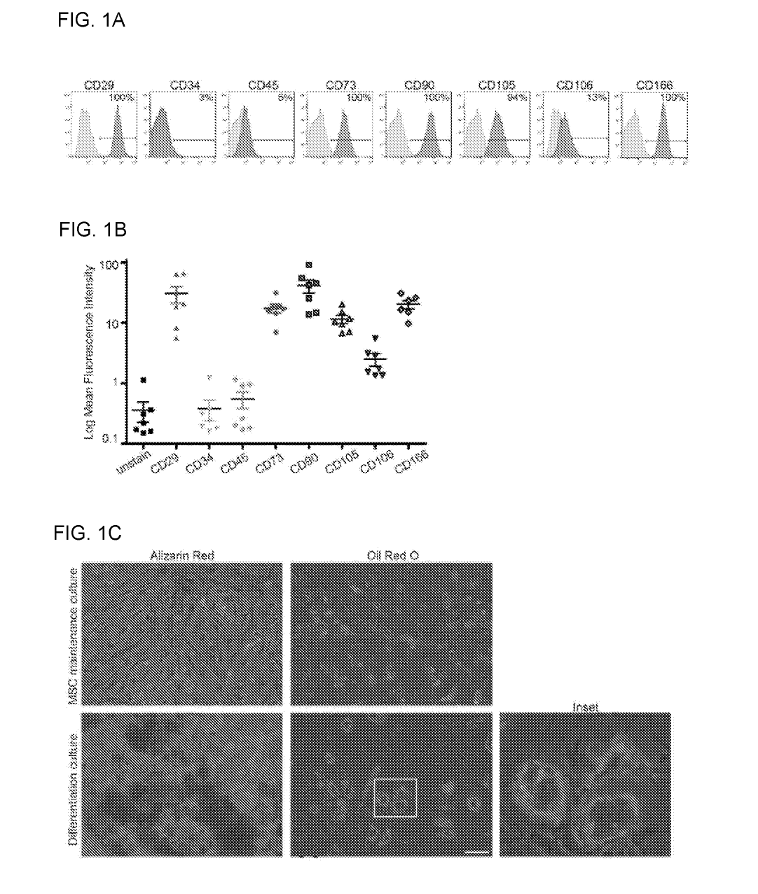

Primary bone marrow-derived MSCs were assessed for a panel of markers, including CD29, CD31, CD34, CD45, CD73, CD90, CD105, CD106, and CD166. The MSCs were uniformly positive for the MSC markers CD29, CD44, CD73, CD90 and CD105, were dim for CD106, and were negative for the endothelial cell marker CD31 and the hematopoietic markers CD34 and CD45 (FIG. 1A). This marker expression profile was consistent across all 7 primary MSC lines tested (FIG. 1B). Two primary MSC lines were tested for the ability to differentiate towards adipogenic and osteogenic lineages (representative images shown in FIG. 1C).

### Example 3

sLe^{x} Surface Expression Peaks 2-3 Days After FUT6-modRNA Transfection and Declines More Slowly than with FTVI Exofucosylation

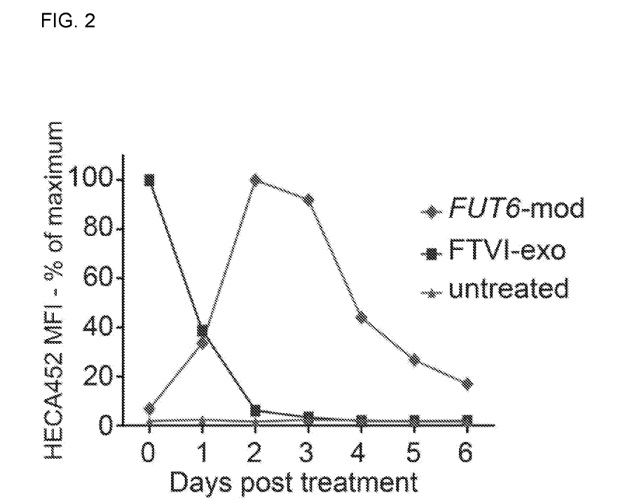

To determine the optimal time point for cell surface E-selectin ligand expression, we compared the kinetics of sLe^{X} surface expression between FTVI exofucosylation and FUT6-modRNA transfection of MSCs by flow cytometry. As expected, the exofucosylated cells had maximal surface sLe^{X} immediately after treatment, decreased to 40% by 24 hours, and returned a baseline level of near zero (i.e., similar to native MSC reactivity) by 48 hours. In contrast, the FUT6-modRNA transfected cells reached maximal cell surface sLe^{X} expression at day 2 post-transfection, with high levels maintained until day 3, followed by gradual decrease thereafter (FIG. 2). Based on these kinetics of induced sLe^{X} expression, all experiments with exofucosylated cells were performed just after treatment, whereas experiments with FUT6-modRNA-transfected cells were performed 2-3 days post-transfection.

### Example 4

sLe^{x} Surface Expression Induced by Intracellular and Extracellular FTVI Fucosylation is Similar and Consistent Across Multiple Primary MSC Lines, and Does Not Alter MSC Properties

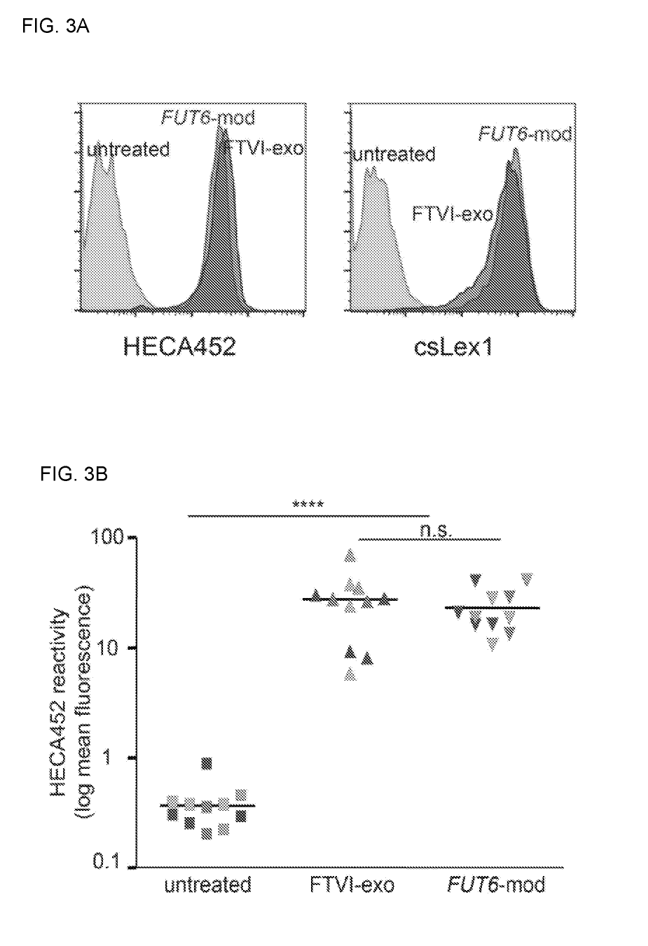

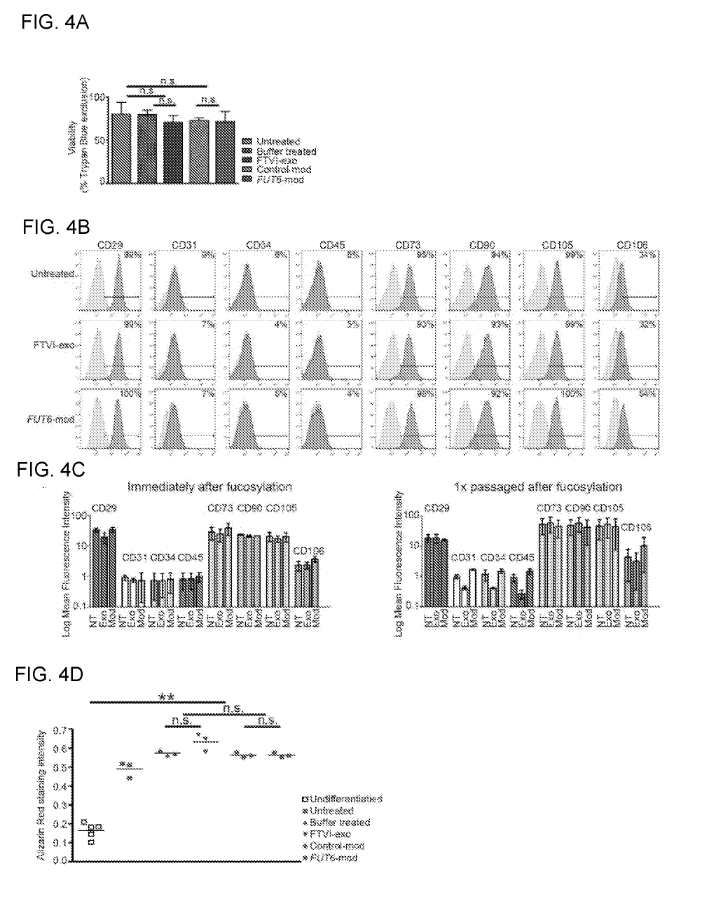

To evaluate the overall extent of fucosylation of cell surface glycans using both methods, we analyzed total cell surface sLe^{X} levels by flow cytometry. This analysis revealed an approximately two-log increase in surface sLe^{X} expression in both intracellularly and extracellularly fucosylated cells (FIG. 3A), results that were confirmed using a second anti-sLe^{X} mAb clone to exclude clone-specific bias (FIG. 3A). Although some variability between MSC primary cultures was observed, on average the increase in cell surface sLe^{X} was similar for both methods when tested in 5 independent primary MSC lines (FIG. 3B). To determine whether either method of FTVI fucosylation affected characteristic MSC biology, we examined several key properties before and after fucosylation (FIG. 4A-FIG. 4D). We observed that MSC viability was not significantly decreased by intracellular or extracellular fucosylation (FIG. 4A), and that a panel of MSC markers did not change, either when measured immediately after fucoslation (FIG. 4B, FIG. 4C) or when cultured for an additional passage (i.e. 5-11 days) (FIG. 4C). Finally, we differentiated the treated cells towards osteoblastic and adipogenic lineages, and no visual differences in differentiation could be observed. Quantification of osteoblastic differentiatiation revealed no significant difference between the intracellularly and extracellularly fucosylated MSCs and their respective controls, and no decrease compared to untreated MSCs (FIG. 4D).

### Example 5

### Comparative Analysis of E-selectin Ligand Glycoproteins Created by Intracellular and Extracellular Fucosylation

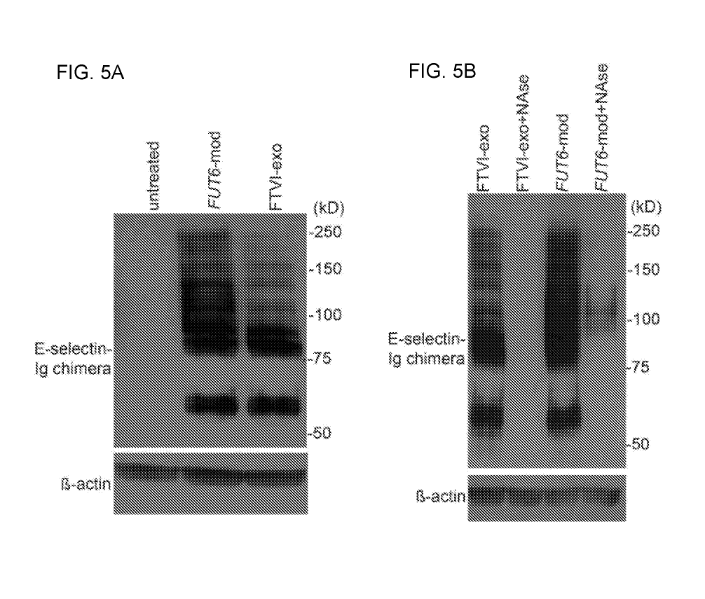

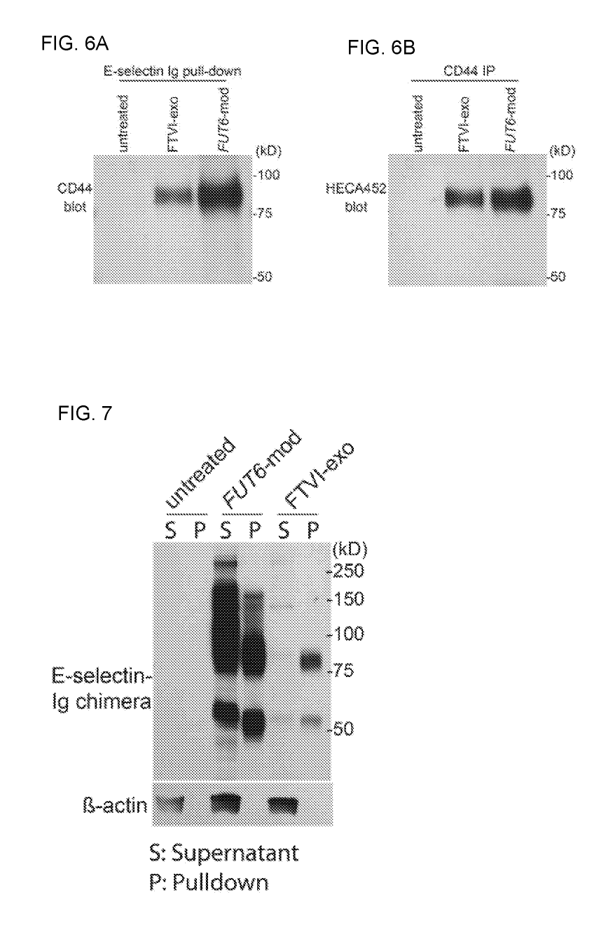

To analyze the identity and cellular localization of the E-selectin ligand glycoproteins created by FUT6-modRNA transfection and FTVI-exofucosylation, we performed western blot using an E-selectin-Ig chimera (E-Ig) as a probe. Lysates from extracellularly fucosylated MSCs exhibited E-Ig reactive bands predominantly at about 85 kD, corresponding in size to HCELL [Sackstein 20098], and about 60 kD, a currently undefined glycoprotein (FIG. 5A). To assess whether the about 85 kD band was indeed HCELL, we immunoprecipitated CD44 and blotted with HECA452, and conversely, isolated E-selectin ligands using E-Ig and blotted with CD44 (FIG. 6A-FIG. 6B). Both HCELL and the about 60 kD band were similarly present in lysates of intracellularly fucosylated MSCs, however, E-Ig reactive bands of larger molecular weights were also observed with much greater intensity in these lysates, suggesting that additional glycoprotein substrates are accessible to fucosylation when FTVI is present in its native intracellular context (FIG. 5A). To determine the cellular localization of the E-Ig reactive proteins, neuraminidase treatment of intact cells was performed to remove sLe^{X} from all cell surface glycoproteins. As expected, no E-Ig reactive glycoproteins remained after neuraminidase treatment of extracellularly fucosylated cells, indicating that all were localized extracellularly. In intracellularly fucosylated cells (day 3), all detectable E-Ig reactive proteins at about 60 kD and about 85 kD were extracellular, however, a portion of the larger E-Ig reactive proteins were still present after neuraminidase treatment, suggesting an intracellular localization (FIG. 5B). This trend was corroborated by cell surface biotinylation experiments, which revealed that the about 60 kD and about 85 kD bands were over-represented within the accessible cell surface proteins compared to the larger E-Ig reactive proteins (FIG. 7).

### Examaple 6

### Intracellular and Extracellular Fucosylation Similarly Enable E-Selectin Ligand-Mediated MSC Capture, Tethering and Rolling Under Fluid Shear Conditions

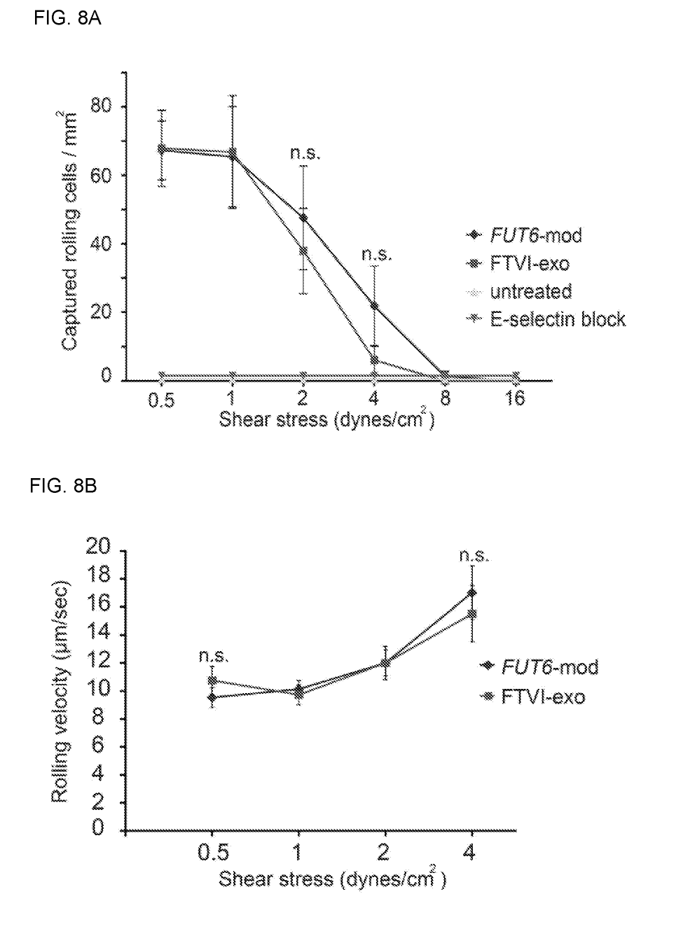

Since sLe^{X} is the critical binding determinant for E-selectin, the dramatic increase in HECA452 and csLex1 reactivity suggests that both intracellular and extracellular fucosylation should enable functional E-selectin binding activity on treated MSCs. To directly assess E-selectin binding activity, we tested the ability of fucosylated and untreated MSCs to capture, tether and roll under fluid shear conditions on HUVEC monolayers stimulated to express E-selectin by treatment with TNFα. Untreated MSCs showed little or no interaction with the stimulated HUVECs at any level of shear stress, consistent with their lack of E-selectin ligand expression. In contrast, both intracellularly and extracellularly fucosylated MSCs were greatly enhanced in their ability to capture, tether and roll on TNFα-stimulated HUVEC monolayers at shear stress levels up to 4 dynes/cm2 (FIG. 8A). No significant difference was observed between extracellularly and extracellularly fucosylated MSCs in the number of rolling cells (FIG. 8A) or rolling velocities (FIG. 8B), suggesting that the similar increased levels of surface sLe^{X} observed by FACS correctly predicted a commensurate functional improvement of the resulting E-selectin ligand activity on the treated MSCs. Non-stimulated HUVECs or HUVECs treated with an anti-E-selectin blocking monoclonal antibody did not support capture, tethering or rolling interactions with fucosylated MSCs, confirming that these interactions were solely E-selectin-mediated.

### Example 7

Both Intracellularly and Extracellularly Fucosylated MSCs Accumulate More Efficiently in Calvarial Bone Marrow than Untreated MSCs

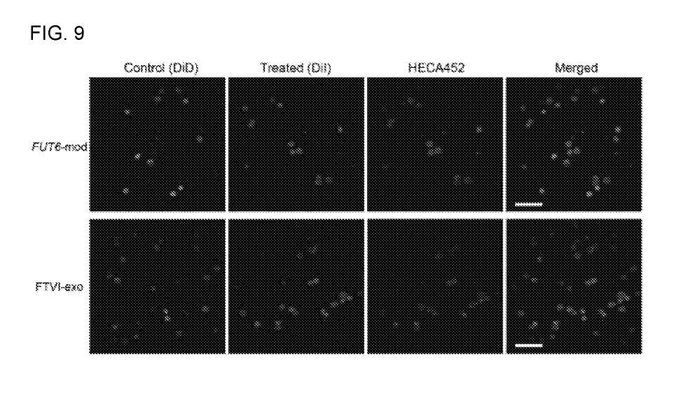

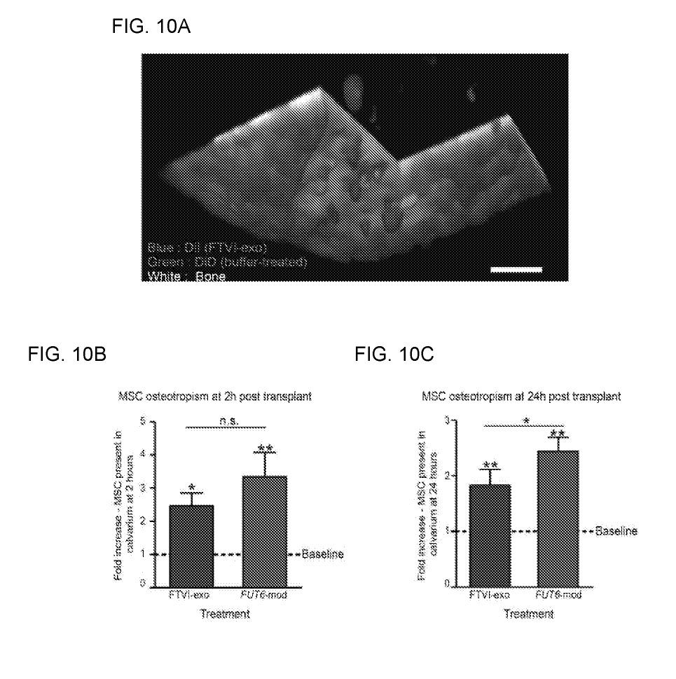

The dramatic increases of cell surface sLe^{X} observed by FACS, of E-Ig reactivity observed by Western blot, and of capture/tethering and rolling on TNFα stimulated HUVECs collectively indicate both intracellular and extracellular fucosylation can create operational E-selectin ligands on MSCs. To determine whether these differences in E-selectin ligands are functionally relevant in vivo, we studied their bone marrow homing properties in vivo using intravital confocal and multiphoton microscopy for cell tracking in the calvarium in murine hosts [Levy 2013, Mortensen 2013]. Intracellularly or extracellularly fucosylated MSCs, together with corresponding non-fucosylated control cells, were each stained with the cell surface dyes DiD or Dil, and 1:1 reciprocal cell mixtures (treated vs control) were prepared. Pairs of mice were transplanted with each cell combination, with the membrane dye combination swapped between the mice in each pair. Aliquots of the cell mixtures injected into each mouse were stained with HECA452 and imaged on a glass slide to confirm the efficacy of fucosylation, and to provide a precise starting ratio (FIG. 9). At approximately 2 hours and again at 24 hours post-transplantation, the calvaria were imaged (FIG. 10A), and DiD and Dil events were counted. Compared to control MSCs, both intracellularly and extracellularly fucosylated MSCs demonstrated significantly increased osteotropism (i.e. accumulation in the bone) at 2 hours post-transplantation (FIG. 10B). When the same mice were imaged at 24 hours post-transplantation, a similar trend was observed, with a further significant increase in cell numbers observed with intracellularly fucosylated MSCs compared to intracellularly fucosylated MSCs (FIG. 10C).

### Example 8

Intracellularly Fucosylated MSCs Demonstrate Significantly Greater Extravasation from Calvarial Vessels Into Bone Marrow Parenchyma at 24 Hours Post-Transplant

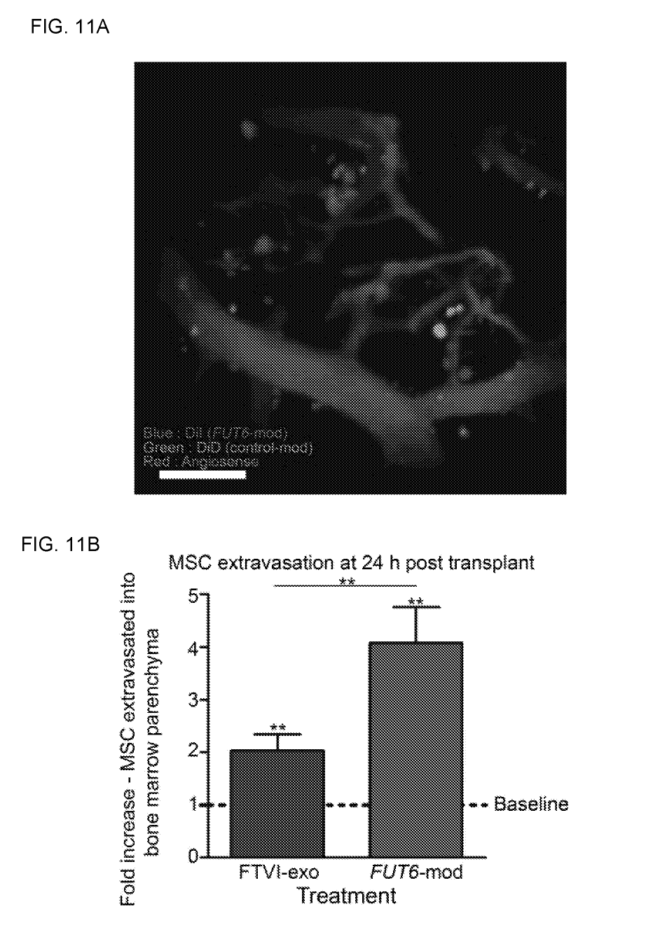

Extravasation of transplanted cells into the marrow parenchyma is prerequisite for engraftment. To evaluate the extent of extravasation, we injected a near-infrared vascular dye (Angiosense 750) to visualize mouse blood vessels and performed multi-stack imaging. We imaged the calvaria at 24 hours post-transplantation to identify Dil and DiD stained cells that had clearly extravasated from the vessels into the surrounding bone marrow space (FIG. 11A), and found that compared to control MSCs, both intracellularly and extracellularly fucosylated MSCs showed significantly more penetration into the marrow parenchyma (FIG. 11B). Furthermore, a clear difference in extravasation was observed between the two treatments, with the intracellularly fucosylated MSCs being two-fold more likely to be extravasated than the extracellularly fucosylated MSCs at 24 hours post-transplantation (FIG. 11 B). These findings suggest that the sustained presence of E-selectin ligands (i.e., beyond day 2) of FUT6-modRNA transduction (FIG. 2) engenders a functional improvement in cell homing and extravasation in an in vivo context.

### Example 9

### Discussion and Conclusion

### Discussion

MSCs represent an avenue of cell therapy that has great potential for clinical impact. There are over 500 past or current registered clinical trials worldwide utilizing MSCs in efforts to treat a broad range of conditions including bone diseases (e.g. osteoporosis, osteogenesis imperfecta), autoimmune diseases (e.g. lupus, multiple sclerosis), and inflammatory diseases (e.g. myocardial infarction, ulcerative colitis) [clinicaltrials.gov, accessed December 2015]. However, while MSC transplantation has been well tolerated, clinical outcomes have generally been disappointing [Griffin 2013, Galipeau 2013]. A major unresolved challenge limiting the clinical efficacy of MSCs is the effective delivery of transplanted MSCs to their intended target site(s). While direct injection of MSCs into injured/diseased organs is possible for some indications, this approach is invasive and can result in collateral tissue damage. Furthermore, for certain organs or for multifocal or systemic conditions, local injection is not feasible, necessitating strategies to optimize vascular delivery of the cells to enable effective site-specific localization.

One of the primary deficiencies that limit MSC homing is their lack of E-selectin ligand expression. Various approaches have been utilized in attempts to engineer MSCs with E-selectin ligands, including covalent peptide linkage to the cell membrane [Cheng 2012], and non-covalent coupling of an E-selectin ligand fusion protein [Lo 2016] or sLe^{X} coated polymer beads [Sarkar 2011]. Arguably however, the most physiologically relevant approach is to harness the power of the human alpha (1,3)-fucosyltransferase enzymes, which by their nature are potent and specific in their ability to convert terminal sialylated lactosamines into sLe^{X}, the canonical selectin binding determinant. We have previously described the use of purified FTVI to exofucosylate the cell surface of MSCs, thus creating the E-selectin ligand HCELL and improving homing to bone [Sackstein 2009]. Exofucosylation has also been employed to enhance selectin-mediated homing and engraftment in other cell types, including umbilical cord hematopoietic cells [Xia 2004, Wan 2013, Popat 2015], regulatory T-cells [Parmar 2015], and neural stem cells [Merzaban 2015]. In contrast, the use of modRNA to generate fucosyltransferase intracellularly in MSCs is new and relatively unexplored. In the only studies to date, human MSCs were co-transfected with modRNAs encoding FTVII, P-selectin glycoprotein ligand-1 (PSGL-1) and the anti-inflammatory cytokine interleukin-10 (IL-10). When these triple-transfected cells were xenotransplanted into mice, a slight enhancement of bone marrow homing was reported, along with a modest improvement in a skin inflammation model [Levy 2013] and an experimental autoimmune encephalomyelitis model [Liao 2016]. However, the nature of the experimental design (i.e. co-transfecting modRNAs to express three genes simultaneously), as well as differences in methodology (different fucosyltransferase, different preclinical models) made it difficult to compare the results with those from other studies employing exofucosylation. In particular, it was not possible to determine from these studies whether the E-selectin ligands created by modRNA transfection are similar in identity and function to those that would be created by the action of extracellular fucosyltransferase, and whether any differences in resulting homing efficiency would be realized.

Our results here indicate that, across multiple primary cultures of human MSCs, intracellular and extracellular fucosylation methods are similarly potent for generation of cell surface E-selectin ligands, as measured by sLe^{X} levels (i.e., as assessed by reactivity to mAb HECA452) and confirmed by assessing E-selectin-mediated capture/tethering/rolling activity under hemodynamic shear conditions on cytokine-stimulated HUVECs. The amount and cellular location of certain E-selectin ligand glycoproteins produced are slightly different between the two methods, with intracellular fucosylation resulting in some additional E-selectin-binding glycoproteins present both intracellularly and extracellularly. Whether the additional intracellular proteins represent novel sLe^{X} bearing glycoproteins that are normally localized inside the cell, or are precursors for export of cell surface presentation (i.e., proteins undergoing further post-translational modifications, stored in granules, or in the process of being shuttled to the cell surface) remains to be determined. The most striking differences between the two methods were the kinetics of E-selectin ligand display on the cell surface. Peak sLe^{X} was observed immediately after extracellular fucosylation with a rapid decline by 1-2 days, whereas, with intracellular fucosylation, sLe^{X} peaked at 48 hours and declined more gradually thereafter. Additionally, while both methods significantly increased osteotropism compared to control MSCs, a larger increase in overall marrow homing and, particularly, in transmigration, was observed for intracellularly fucosylated cells at 24 hours post-transplant in vivo. Considering the fact that MSCs were injected immediately after exofucosylation or day 2 post-modRNA transfection, it is likely that the markedly different levels of E-selectin ligands remaining on the cell surface 24 hours later contributed to these differences. Additional studies are warranted to determine the molecular basis of this effect, but it could also relate to heightened glycan accepter accessibility in the Golgi and/or differences in membrane distribution of intracellularly glycosylated products.

Our findings are important for informing future clinical applications using human MSCs and other cells of interest (e.g., other types of stem cells, of tissue progenitor cells, or of leukocytes). Both FTVI exofucosylation and FUT6-modRNA transfection are ideal glycoengineering strategies as they are simple, transient, and non-integrative. In addition to the longer duration of E-selectin ligand expression after intracellular glycosylation and the associated improvement in homing and transmigration properties described here, a practical advantage of this approach is that the FTVI enzyme and GDP-Fucose are cell products, thereby eliminating the effort and expense associated with the purification of soluble recombinant enzyme and synthesis of GDP-Fucose. Furthermore, since the FTVI enzyme is localized in its native cellular context (i.e., embedded in the Golgi membrane), additional accepter substrates are accessible for fucosylation. On the other hand, practical advantages to extracellular fucosylation include the rapidity of the treatment (thus avoiding further culture of the cells), the avoidance of potential disruption of Golgi glycosylation networks, and the elimination of risks involved with introducing nucleic acids into cells, including, but not limited to, activation of cellular antiviral defense mechanisms. Furthermore, when considering fucosylation of other (i.e., non-MSC) clinically-relevant cells, exofucosylation is easily applicable to any cell type bearing sialylated lactosamines on its cell surface, in contrast to intracellular fucosylation (or other intracellular glycosyltransferase modifications) which is limited to those cell types that are readily transfectable with nucleic acids (such as modRNA) that encode fucosyltransferase(s) needed to enforce cell surface sLe^{X} expression or where nucleic acids encoding relevant fucosyltransferase(s) needed to enforce cell surface sLe^{X} expression can be introduced by other means (e.g., transduced via viral vectors). However, in those cells that can be transfected or transduced, the introduction of relevant nucleic acid sequences encoding glycosyltransferase(s) needed to enforce cell surface sLe^{X} expression could be combined with cell surface (extracellular) fucosylation to engender and/or augment cell surface sLe^{X} expression. Such combinatorial strategies are encompassed within the scope of this invention.

We note that intracellular fucosylation via the introduction of fucosyltransferase-encoding nucleic acid (e.g., modRNA) could be combined with a fucosyltransferase-mediated exofucosylation process to yield a substantially higher (and prolonged) expression of E-selectin ligand activity on cells. In many cases, introduction of nucleic acid that encodes a glycosyltransferase to enforce expression of cell surface sLe^{X} may be useful in a diverse population of clinically relevant cell types, including, e.g., embryonic stem cells, adult stem cells and induced pluripotent stem cells (iPSCs). Adult stem cells include stem cells obtained from any clinically relevant site including from bone marrow, cord blood, adipose tissue, placental tissue, skin, muscle, liver, pancreas, neuronal tissue, tissues of the eye, and, indeed, from any cell type derived from ectodermal, endodermal or mesenchymal cell lineages. Therefore, depending on the specific clinical application(s), one might favor utility of the intracellular or the extracellular fucosylation approach.

It is now clear that maximizing E-selectin interactions via fucosylation is a valid strategy for improving osteotropism and may be useful in treating a wide range of medical disorders, including but not limited to inflammatory disorders (e.g., autoimmune diseases such as diabetes and rheumatoid arthritis), degenerative diseases (e.g., osteoporosis), cardiovascular diseases, ischemic conditions, and cancer. However, MSCs and other cells of interest (e.g., other types of stem cells, tissue progenitors or leukocytes) can also be modified in other ways to further improve homing and/or differentiation into relevant cell types. For example, efforts have been made to improve bone surface retention of MSCs by affixing alendronate to MSCs [Yao 2013], improving cell migration into the tissue by upregulating expression of chemokine receptors (such as CXCR4) [Wynn 2004, Shi 2007, Jones 2012], and improving firm adhesion and differentiation to bone by increasing integrin levels or activity [Kumar 2007, Srouji 2012, Hamidouche 2009]. It seems reasonable that future translational efforts could seek to combine multiple homing and differentiation approaches in a specific and step-wise fashion to enhance engagement of MSC or of other relevant cells at each stage of the homing, engraftment and differentiation process. Fucosylation could thus be used as an important aspect of a combinatorial approach to maximize the clinical utility of all cell-based therapeutics.

We further believe that maximizing E-selectin interactions via fucosylation, particularly via the modRNA process or other means of introduction of nucleic acid sequences encoding a relevant a(1,3)-fucosyltransferase, is likely a valid strategy for treating or improving a number of medical disorders including, but not limited to those initiated by direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, SIRS, etc.), neoplasia (e.g., breast cancer, lung cancer, lymphoma, etc.), immunologic/autoimmune conditions (e.g., graft vs. host disease, multiple sclerosis, diabetes, inflammatory bowel disease, lupus erythematosus, rheumatoid arthritis, psoriasis, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.).

The present disclosure is additionally directed to the treatment of a disease, disorder, or medical condition wherein E-selectin is expressed in endothelial beds of the affected tissue(s) and/or L-selectin-expressing leukocytes have infiltrated/accumulated in the affected tissue(s) by maximizing E-selectin interactions via fucosylation, particularly using the modRNA process. As discussed above, E-selectin and L-selectin each bind to sialylated, fucosylated carbohydrates, and enforced expression of these sialofucosylated glycan structures on the cell surface serves to program binding to these selectins. Accordingly, the disclosure describes methods to enhance homing to target tissue(s) by augmenting the expression of E-selectin ligands on administered cells; additionally, in describing methods to enhance expression of potent E-selectin and L-selectin ligands (such as HCELL) on administered cells to promote adherence to E-selectin on vascular endothelial cells and/or of L-selectin on tissue-infiltrating leukocytes within affected tissue(s), the disclosure provides a means to augment colonization/lodgement of the cells within relevant tissue microenvironments where biologic effects are intended. In general, the methods described herein have utility in improving the outcome of any cell-based therapeutic approach, be it in immunotherapy applications (e.g., administration of culture-expanded antigen-specific T cells and/or culture expanded NK cells for cancer or infectious disease applications, administration of culture-expanded chimeric antigen receptor (CAR) T cells, administration of antigen-pulsed dendritic cells, etc.), immunomodulatory/immunosuppressive therapeutic applications (e.g., administration of culture-expanded regulatory T cells (Tregs), administration of antigen-pulsed dendritic cells, administration of mesenchymal stem cells, administration of culture-expanded NKT cells, etc.), or tissue repair/regenerative medicine applications (e.g., use of stem and/or progenitor cells or other tissue-reparative cells for tissue regeneration/restoration; use of culture-expanded stem cells and/or culture-expanded progenitor cells for tissue regeneration/restoration). With utility in regenerative medicine applications, it is understood that administered cells may themselves contribute to regenerate the target tissue by way of long-term engraftment (with attendant proliferation/differentiation) yielding tissue-specific cells (e.g., such as in transplantation of hematopoietic stem cells for blood cell production) and/or may deliver a tissue restorative/reparative effect without long-term engraftment or differentiation into tissue-resident cells (e.g., via delivery of trophic effects that stimulate resident stem/progenitors to repair the injured tissue(s) and/or by dampening inflammatory processes that promote injury and impede repair). All applications for all indications described herein can be used alone or in combination with enhancing agents (e.g., growth factors, tissue scaffolds, etc.). Any and all diseases, disorders, or medical conditions having associated inflammation (e.g., acute and/or chronic), tissue injury/damage or neoplastic conditions may be treated in accordance with the methods described herein, including, but not limited to those initiated by direct tissue injury (e.g., burns, trauma, bone fracture, bone deformities, decubitus ulcers, etc.), ischemic/vascular events (e.g., myocardial infarct, stroke, shock, hemorrhage, coagulopathy, etc.), infections (e.g., cellulitis, pneumonia, meningitis, cystitis, sepsis, SIRS, etc.), neoplasia (e.g., breast cancer, lung cancer, prostate cancer, renal cell cancer, lymphoma, leukemia, etc.), immunologic/autoimmune conditions (e.g., acute or chronic GVHD, multiple sclerosis, diabetes, inflammatory bowel disease (e.g., Crohn's disease, ulcerative colitis), rheumatoid arthritis, psoriasis, etc.), degenerative diseases (e.g., osteoporosis, osteoarthritis, spinal disc degeneration, Alzheimer's disease, atherosclerosis, etc.), congenital/genetic diseases (e.g., epidermolysis bullosa, osteogenesis imperfecta, muscular dystrophies, lysosomal storage diseases, Huntington's disease, etc.), adverse drug effects (e.g., chemotherapy-induced tissue/organ toxicity, radiotherapy toxicity, drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). Other general and specific diseases, disorders, or medical conditions that may be treated in accordance with the methods described herein include, but are not limited to:
Acute Leukemias, e.g., Acute Biphenotypic Leukemia, Acute Lymphocytic Leukemia (ALL), Acute Myelogenous Leukemia (AML), and Acute Undifferentiated Leukemia;
Myelodysplastic Syndromes, e.g., Amyloidosis Chronic Myelomonocytic Leukemia (CMML), Refractory Anemia (RA), Refractory Anemia with Excess Blasts (RAEB), Refractory Anemia with Excess Blasts in Transformation (RAEB-T), and Refractory Anemia with Ringed Sideroblasts (RARS);
Myeloproliferative Disorders, e.g., Acute Myelofibrosis, Agnogenic Myeloid Metaplasia (Myelofibrosis), Essential Thrombocythemia, chronic myelogenous leukemia, and Polycythemia Vera;
Phagocyte Disorders, e.g., Chediak-Higashi Syndrome, Chronic Granulomatous Disease, Leukocyte adhesion deficiencies, myeloperoxidase deficiency, Neutrophil Actin Deficiency, and Reticular Dysgenesis;
Lysosomal Storage Diseases, e.g., Adrenoleukodystrophy, Alpha Mannosidosis, Gaucher's Disease, Hunter's Syndrome (MPS-II), Hurler's Syndrome (MPS-IH), Krabbe Disease, Maroteaux-Lamy Syndrome (MPS-VI), Metachromatic Leukodystrophy, Morquio Syndrome (MPS-IV), Mucolipidosis II (I-cell Disease), Mucopolysaccharidoses (MPS), Niemann-Pick Disease, Sanfilippo Syndrome (MPS-III), Scheie Syndrome (MPS-IS), Sly Syndrome, Beta-Glucuronidase Deficiency (MPS-VII), and Wolman Disease;
Inherited Erythrocyte Abnormalities, _ e.g., Beta Thalassemia, Blackfan-Diamond Anemia, Pure Red Cell Aplasia, and Sickle Cell Disease;
Inherited Platelet Abnormalities, e.g., Amegakaryocytosis/Congenital Thrombocytopenia, Gray platelet syndrome;
Solid organ malignancies, e.g., Brain Tumors, Ewing Sarcoma, Neuroblastoma, Ovarian Cancer, Renal Cell Carcinoma, Lung Cancers, Breast cancers, Gastric cancers, Esophageal cancers, Skin cancers, Oral cancers, Endocrine cancers, Liver cancers, Biliary system cancers, Pancreatic cancer, Prostate Cancer, and Testicular Cancer;
Other Applications, e.g., Bone Marrow Transplants, Heart Disease (myocardial infarction), Liver Disease, Muscular Dystrophy, Alzheimer's Disease, Parkinson's Disease, Spinal Cord Injury, Spinal disc disease/degeneration, Bone disease, Bone fracture, Stroke, Peripheral Vascular Disease, Head trauma, Bullous diseases, Mitochondrial diseases, Ex vivo and In vivo expanded stem and progenitor cell populations, In vitro fertilization application and enhancement, Hematopoietic Rescue Situations (Intense Chemo/Radiation), Stem cells and progenitor cells derived from various tissues sources, Application in humans and animals, and Limb regeneration, reconstructive surgical procedures/indications, alone or in combination with enhancing agents;
Chronic Leukemias, e.g., Chronic Lymphocytic Leukemia (CLL), Chronic Myelogenous Leukemia (CML), Juvenile Chronic Myelogenous Leukemia (JCML), and Juvenile Myelomonocytic Leukemia (JMML), Stem Cell Disorders, e.g., Aplastic Anemia (Severe), Congenital Cytopenia, Dyskeratosis Congenita, Fanconi Anemia, and Paroxysmal Nocturnal Hemoglobinuria (PNH);
Lymphoproliferative Disorders, e.g., Hodgkin's Disease, Non-Hodgkin's Lymphomas, and Prolymphocytic Leukemia;
Histiocytic Disorders, e.g., Familial Erythrophagocytic Lymphohistiocytosis, Hemophagocytosis, Hemophagocytic Lymphohistiocytosis, Histiocytosis-X, and Langerhans' Cell Histiocytosis;
Congenital (Inherited) Immune System Disorders, e.g., Absence of T and B Cells, Absence of T Cells, Normal B Cell SCID, Ataxia-Telangiectasia, Bare Lymphocyte Syndrome, Common Variable Immunodeficiency, DiGeorge Syndrome, Kostmann Syndrome, Leukocyte Adhesion Deficiency, Omenn's Syndrome, Severe Combined Immunodeficiency (SCID), SCID with Adenosine Deaminase Deficiency, Wiskott-Aldrich Syndrome, and X-Linked Lymphoproliferative Disorder;
Other Inherited Disorders, e.g., Cartilage-Hair Hypoplasia, Ceroid Lipofuscinosis, Congenital Erythropoietic Porphyria, Familial Mediterranean Fever, Glanzmann Thrombasthenia, Lesch-Nyhan Syndrome, Osteopetrosis, and Sandhoff Disease;
Plasma Cell Disorders, e.g., Multiple Myeloma, Plasma Cell Leukemia, and Waldenstrom's Macroglobulinemia;
Autoimmune Diseases, e.g., Multiple Sclerosis, Rheumatoid Arthritis, Systemic Lupus Erythematosus, Scleroderma, Ankylosing spondylitis, Diabetes Mellitus, and Inflammatory Bowel Diseases;
Articular and skeletal diseases/conditions, e.g., disc degeneration, synovial disease, cartilage degeneration, cartilage trauma, cartilage tears, arthritis, bone fractures, bone deformities, bone reconstruction, osteogenesis imperfecta, congenital bone diseases/conditions, genetic bone diseases/conditions, osteoporosis. Osteopetrosis, hypophosphatasia, metabolic bone disease, etc.; and
Skin/soft tissue diseases and conditions such as bullous diseases, psoriasis, eczema, epidermolysis bullosa, ulcerative skin conditions, soft tissue deformities (including post-surgical skin and soft tissue deformities), plastic surgery/reconstructive surgery indications, etc.

In general, associated inflammation symptoms include, without limitation, fever, pain, edema, hyperemia, erythema, bruising, tenderness, stiffness, swollenness, chills, respiratory distress, hypotension, hypertension, stuffy nose, stuffy head, breathing problems, fluid retention, blood clots, loss of appetite, weight loss, polyuria, nocturia, anuria, dyspnea, dyspnea on exertion, muscle weakness, sensory changes, increased heart rate, decreased heart rate, arrythmias, polydipsia, formation of granulomas, fibrinous, pus, non-viscous serous fluid, or ulcers. The actual symptoms associated with an acute and/or chronic inflammation are well known and can be determined by a person of ordinary skill in the art by taking into account factors, including, without limitation, the location of the inflammation, the cause of the inflammation, the severity of the inflammation, the tissue or organ affected, and the associated disorder.

Specific patterns of acute and/or chronic inflammation are seen during particular situations that arise in the body, such as when inflammation occurs on an epithelial surface, or pyogenic bacteria are involved. For example, granulomatous inflammation is an inflammation resulting from the formation of granulomas arising from a limited but diverse number of diseases, which include, without limitation, tuberculosis, leprosy, sarcoidosis, and syphilis. Purulent inflammation is an inflammation resulting in large amount of pus, which consists of neutrophils, dead cells, and fluid. Infection by pyogenic bacteria such as staphylococci is characteristic of this kind of inflammation. Serous inflammation is an inflammation resulting from copious effusion of non-viscous serous fluid, commonly produced by mesothelial cells of serous membranes, but may be derived from blood plasma. Skin blisters exemplify this pattern of inflammation.

Ulcerative inflammation is an inflammation resulting from the necrotic loss of tissue from the epithelial surface, exposing lower layers and forming an ulcer.

An acute and/or chronic inflammation symptom can be associated with a large, unrelated group of disorders which underlay a variety of diseases and disorders. The immune system is often involved with acute and/or chronic inflammatory disorders, demonstrated in both allergic reactions, arthritic conditions, and some myopathies, with many immune system disorders resulting in abnormal inflammation. Non-immune diseases with etiological origins in acute and/or chronic inflammatory processes include cancer, atherosclerosis, and ischaemic heart disease. Non-limiting examples of disorders exhibiting acute and/or chronic inflammation as a symptom include, without limitation, acne, acid reflux/heartburn, age related macular degeneration (AMD), allergy, allergic rhinitis, Alzheimer's disease, amyotrophic lateral sclerosis, anemia, appendicitis, arteritis, arthritis, asthma, atherosclerosis, autoimmune disorders, balanitis, blepharitis, bronchiolitis, bronchitis, a bullous pemphigoid, burn, bursitis, cancer, cardiac arrest, carditis, celiac disease, cellulitis, cervicitis, cholangitis, cholecystitis, chorioamnionitis, chronic obstructive pulmonary disease (COPD) (and/or acute exacerbations thereof), cirrhosis, colitis, congestive heart failure, conjunctivitis, drug-induced tissue injury (e.g., cyclophosphamide-induced cystitis), cystic fibrosis, cystitis, common cold, dacryoadenitis, decubitus ulcers, dementia, dermatitis, dermatomyositis, diabetes, diabetic neuropathy, diabetic retinopathy, diabetic nephropathy, diabetic ulcer, digestive system disease, eczema, emphysema, encephalitis, endocarditis, endocrinopathies, endometritis, enteritis, enterocolitis, epicondylitis, epididymitis, fasciitis, fibromyalgia, fibrosis, fibrositis, gastritis, gastroenteritis, gingivitis, glomerulonephritis, glossitis, heart disease, heart valve dysfunction, hepatitis, hidradenitis suppurativa, Huntington's disease, hyperlipidemic pancreatitis, hypertension, ileitis, infection, inflammatory bowel disease, inflammatory cardiomegaly, inflammatory neuropathy, insulin resistance, interstitial cystitis, interstitial nephritis, iritis, ischemia, ischemic heart disease, keratitis, keratoconjunctivitis, laryngitis, lupus nephritis, macular degeneration, mastitis, mastoiditis, meningitis, metabolic syndrome (syndrome X), a migraine, mucositis, multiple sclerosis, myelitis, myocarditis, myositis, nephritis, neuronitis, non-alcoholic steatohepatitis, obesity, omphalitis, oophoritis, orchitis, osteochondritis, osteopenia, osteomyelitis, osteoporosis, osteitis, otitis, pancreatitis, Parkinson's disease, parotitis, pelvic inflammatory disease, pemphigus vularis, pericarditis, peritonitis, pharyngitis, phlebitis, pleuritis, pneumonitis, polycystic nephritis, proctitis, prostatitis, psoriasis, pulpitis, pyelonephritis, pylephlebitis, radiation-induced injury, renal failure, reperfusion injury, retinitis, rheumatic fever, rhinitis, salpingitis, sarcoidosis, sialadenitis, sinusitis, spastic colon, stasis dermatitis, stenosis, stomatitis, stroke, surgical complication, synovitis, tendonitis, tendinosis, tenosynovitis, thrombophlebitis, thyroiditis, tonsillitis, trauma, traumatic brain injury, transplant rejection, trigonitis, tuberculosis, tumor, ulcers, urethritis, ursitis, uveitis, vaginitis, vasculitis, and vulvitis.

General categories of diseases, disorders, and trauma that can result in or otherwise cause acute and/or chronic inflammation include, but are not limited to genetic diseases, neoplasias, direct tissue injury, autoimmune diseases, infectious diseases, vascular diseases/complications (e.g., ischemia/reperfusion injury), iatrogenic causes (e.g. drug adverse effects, radiation injury, etc.), and allergic manifestations.

In one embodiment, an acute and/or chronic inflammation comprises a tissue inflammation. In general, tissue inflammation is an acute and/or chronic inflammation that is confined to a particular tissue or organ. Thus, for example, a tissue inflammation may comprise a skin inflammation, a muscle inflammation, a tendon inflammation, a ligament inflammation, a bone inflammation, a cartilage/joint inflammation, a lung inflammation, a heart inflammation, a liver inflammation, a gall bladder inflammation, a pancreatic inflammation, a kidney inflammation, a bladder inflammation, an gum inflammation, an esophageal inflammation, a stomach inflammation, an intestinal inflammation, an anal inflammation, a rectal inflammation, a vessel inflammation, a vaginal inflammation, a uterine inflammation, a testicular inflammation, a penile inflammation, a vulvar inflammation, a neuron inflammation, an oral inflammation, an ocular inflammation, an aural inflammation, a brain inflammation, a ventricular/meningial inflammation and/or inflammation involving central or peripheral nervous system cells/elements.

In another embodiment, an acute and/or chronic inflammation comprises a systemic inflammation. Although the processes involved are similar if not identical to tissue inflammation, systemic inflammation is not confined to a particular tissue but rather involves multiple sites within the body, involving the epithelium, endothelium, nervous tissues, serosal surfaces and organ systems. When it is due to infection, the term sepsis can be used, with bacteremia being applied specifically for bacterial sepsis and viremia specifically to viral sepsis. Vasodilation and organ dysfunction are serious problems associated with widespread infection that may lead to septic shock and death.

In another embodiment, an acute and/or chronic inflammation is induced by an arthritis. Arthritis includes a group of conditions involving damage to the joints of the body due to the inflammation of the synovium including, for example, osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also commonly referred to as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

In another embodiment, an acute and/or chronic inflammation is induced by an autoimmune disorder. Autoimmune diseases can be broadly divided into systemic and organ-specific autoimmune disorders, depending on the principal clinico-pathologic features of each disease. Systemic autoimmune diseases include, for example, systemic lupus erythematosus (SLE), Sjogren's syndrome, Scleroderma, rheumatoid arthritis and polymyositis. Local autoimmune diseases may be endocrinologic (Diabetes Mellitus Type 1, Hashimoto's thyroiditis, Addison's disease, etc.), dermatologic (pemphigus vulgaris), hematologic (autoimmune haemolytic anemia), neural (multiple sclerosis) or can involve virtually any circumscribed mass of body tissue. Types of autoimmune disorders include, without limitation, acute disseminated encephalomyelitis (ADEM), Addison's disease, an allergy or sensitivity, amyotrophic lateral sclerosis (ALS), anti-phospholipid antibody syndrome (APS), arthritis, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD) (including acute exacerbations thereof), diabetes mellitus type 1 (IDDM), endometriosis, fibromyalgia, Goodpasture's syndrome, Graves' disease, Guillain-Barre syndrome (GBS), Hashimoto's thyroiditis, hidradenitis suppurativa, idiopathic thrombocytopenic purpura, inflammatory bowel disease (IBD), interstitial cystitis, lupus (including discoid lupus erythematosus, drug-induced lupus erythematosus, lupus nephritis, neonatal lupus, subacute cutaneous lupus erythematosus and systemic lupus erythematosus), morphea, multiple sclerosis (MS), myasthenia gravis, myopathies, narcolepsy, neuromyotonia, pemphigus vulgaris, pernicious anaemia, primary biliary cirrhosis, recurrent disseminated encephalomyelitis (multiphasic disseminated encephalomyelitis), rheumatic fever, schizophrenia, scleroderma, Sjogren's syndrome, tenosynovitis, vasculitis, and vitiligo. In one particular embodiment, the acute and/or chronic inflammation results from or is otherwise caused by diabetes in the subject. In another particular embodiment, the acute and/or chronic inflammation results from or is otherwise caused by multiple sclerosis in the subject.

In another embodiment, an acute and/or chronic inflammation is induced by a myopathy. In general, myopathies are caused when the immune system inappropriately attacks components of the muscle, leading to inflammation in the muscle. A myopathy includes, for example, an inflammatory myopathy and an auto-immune myopathy. Myopathies include, for example, dermatomyositis, inclusion body myositis, and polymyositis.

In another embodiment, an acute and/or chronic inflammation is induced by a vasculitis. Vasculitis is a varied group of disorders featuring inflammation of a vessel wall including lymphatic vessels and blood vessels like veins (phlebitis), arteries (arteritis) and capillaries due to leukocyte migration and resultant damage. The inflammation may affect any size blood vessel, anywhere in the body. It may affect either arteries and/or veins. The inflammation may be focal, meaning that it affects a single location within a vessel, or it may be widespread, with areas of inflammation scattered throughout a particular organ or tissue, or even affecting more than one organ system in the body. Vasculitis include, without limitation, Buerger's disease (thromboangiitis obliterans), cerebral vasculitis (central nervous system vasculitis), ANCA-associated vasculitis, Churg-Strauss arteritis, cryoglobulinemia, essential cryoglobulinemic vasculitis, giant cell (temporal) arteritis, Golfer's vasculitis, Henoch-Schonlein purpura, hypersensitivity vasculitis (allergic vasculitis), Kawasaki disease, microscopic polyarteritis/polyangiitis, polyarteritis nodosa, polymyalgia rheumatica (PMR), rheumatoid vasculitis, Takayasu arteritis, Wegener's granulomatosis, and vasculitis secondary to connective tissue disorders like systemic lupus erythematosus (SLE), rheumatoid arthritis (RA), relapsing polychondritis, Behcet's disease, or other connective tissue disorders, vasculitis secondary to viral infection.

In another embodiment, an acute and/or chronic inflammation is induced by a skin disorder. Skin disorders include, for example, an acne, including acne vulgaris, a bullous phemigoid, a dermatitis, including atopic dermatitis and acute and/or chronic actinic dermatitis, an eczema-like atopic eczema, contact eczema, xerotic eczema, seborrhoeic dermatitis, dyshidrosis, discoid eczema, venous eczema, dermatitis, dermatitis herpetiformis, neurodermatitis, and autoeczematization, and stasis dermatitis, diabetic skin complications, hidradenitis suppurativa, lichen planus, psoriasis including plaqure psoriasis, nail psoriasis, guttate psoriasis, scalp psoriasis, inverse psoriasis, pustular psoriasis, erythrodermis psoriasis, and psoriatic arthritis, rosacea and scleroderma including morphea, ulcers.

In another embodiment, an acute and/or chronic inflammation is induced by a gastrointestinal disorder. A gastrointestinal disorder includes, for example, irritable bowel disease (IBD), an inflammatory bowel disease including Crohn's disease and an ulcerative colitis like ulcerative proctitis, left-sided colitis, pancolitis, and fulminant colitis.

In another embodiment, an acute and/or chronic inflammation is induced by a cardiovascular disease. When LDL cholesterol becomes embedded in arterial walls, it can invoke an immune response. Acute and/or chronic inflammation eventually can damage the arteries, which can cause them to burst. In general, cardiovascular disease is any of a number of specific diseases that affect the heart itself and/or the blood vessel system, especially the veins and arteries leading to and from the heart. There are over 60 types of cardiovascular disorders including, for example, a hypertension, endocarditis, myocarditis, heart valve dysfunction, congestive heart failure, myocardial infarction, a diabetic cardiac conditions, blood vessel inflammation like arteritis, phlebitis, vasculitis; arterial occlusive disease like arteriosclerosis and stenosis, inflammatory cardiomegaly, a peripheral arterial disease; an aneurysm; an embolism; a dissection; a pseudoaneurysm; a vascular malformation; a vascular nevus; a thrombosis; a thrombophlebitis; a varicose veins; a stroke. Symptoms of a cardiovascular disorder affecting the heart include, without limitation, chest pain or chest discomfort (angina), pain in one or both arms, the left shoulder, neck, jaw, or back, shortness of breath, dizziness, faster heartbeats, nausea, abnormal heartbeats, feeling fatigued. Symptoms of a cardiovascular disorder affecting the brain include, without limitation, sudden numbness or weakness of the face, arm, or leg, especially on one side of the body, sudden confusion or trouble speaking or understanding speech, sudden trouble seeing in one or both eyes, sudden dizziness, difficulty walking, or loss of balance or coordination, sudden severe headache with no known cause. Symptoms of a cardiovascular disorder affecting the legs, pelvis and/or arm include, without limitation, claudication, which is a pain, ache, or cramp in the muscles, and cold or numb feeling in the feet or toes, especially at night.

In another embodiment, an acute and/or chronic inflammation is induced by a cancer. In general, inflammation orchestrates the microenvironment around tumors, contributing to proliferation, survival and migration. For example, fibrinous inflammation results from a large increase in vascular permeability which allows fibrin to pass through the blood vessels. If an appropriate procoagulative stimulus is present, such as cancer cells, a fibrinous exudate is deposited. This is commonly seen in serous cavities, where the conversion of fibrinous exudate into a scar can occur between serous membranes, limiting their function. In another example, a cancer is an inflammatory cancer like a NF-κB-driven inflammatory cancer.

In another embodiment, an acute and/or chronic inflammation is a pharmacologically-induced inflammation. Certain drugs or exogenic chemical compounds, including deficiencies in key vitamins and minerals, are known to effect inflammation. For example, Vitamin A deficiency causes an increase in an inflammatory response, Vitamin C deficiency causes connective tissue disease, and Vitamin D deficiency leads to osteoporosis. Certain pharmacologic agents can induce inflammatory complications, e.g., drug-induced hepatitis. Certain illicit drugs such as cocaine and ecstasy may exert some of their detrimental effects by activating transcription factors intimately involved with inflammation (e.g., NF-κB). Radiation therapy can induce pulmonary toxicity, burns, myocarditis, mucositis, and other tissue injuries depending on site of exposure and dose.

In another embodiment, an acute and/or chronic inflammation is induced by an infection. An infectious organism can escape the confines of the immediate tissue via the circulatory system or lymphatic system, where it may spread to other parts of the body. If an organism is not contained by the actions of acute inflammation it may gain access to the lymphatic system via nearby lymph vessels. An infection of the lymph vessels is known as lymphangitis, and infection of a lymph node is known as lymphadenitis. A pathogen can gain access to the bloodstream through lymphatic drainage into the circulatory system. Infections include, without limitation, bacterial cystitis, bacterial encephalitis, pandemic influenza, viral encephalitis, and viral hepatitis (A, B and C).

In another embodiment, an acute and/or chronic inflammation is induced by a tissue or organ injury. Tissue or organ injuries include, without limitation, a burn, a laceration, a wound, a puncture, or a trauma.

In another embodiment, an acute and/or chronic inflammation is induced by a transplant rejection. Transplant rejection occurs when a transplanted organ or tissue is not accepted by the body of the transplant recipient because the immune system of the recipient attacks the transplanted organ or tissue. An adaptive immune response, transplant rejection is mediated through both T-cell-mediated and humoral immune (antibodies) mechanisms. A transplant rejection can be classified as a hyperacute rejection, an acute rejection, or a chronic rejection. Acute and/or chronic rejection of a transplanted organ or tissue is where the rejection is due to a poorly understood acute and/or chronic inflammatory and immune response against the transplanted tissue. Also included as transplant rejection is graft-versus-host disease (GVHD), either acute or chronic GVHD. GVHD is a common complication of allogeneic bone marrow transplantation in which functional immune cells in the transplanted marrow recognize the recipient as “foreign” and mount an immunologic attack. It can also take place in a blood transfusion under certain circumstances. GVHD is divided into acute and chronic forms. Acute and chronic GVHD appear to involve different immune cell subsets, different cytokine profiles, somewhat different host targets, and respond differently to treatment. In another embodiment, an acute and/or chronic inflammation is induced by a Th1-mediated inflammatory disease.

In a well-functioning immune system, an immune response should result in a well-balanced pro-inflammatory Th1 response and anti-inflammatory Th2 response that is suited to address the immune challenge. Generally speaking, once a pro-inflammatory Th1 response is initiated, the body relies on the anti-inflammatory response invoked by a Th2 response to counteract this Th1 response. This counteractive response includes the release of Th2 type cytokines such as, e.g., IL-4, IL-5, and IL-13 which are associated with the promotion of IgE and eosinophilic responses in atopy, and also IL-10, which has an anti-inflammatory response. A Th1-mediated inflammatory disease involves an excessive pro-inflammatory response produced by Th1 cells that leads to acute and/or chronic inflammation. The Th1-mediated disease may be virally, bacterially or chemically (e.g., environmentally) induced. For example, a virus causing the Th1-mediated disease may cause a chronic or acute infection, which may cause a respiratory disorder or influenza.

In another embodiment, an acute and/or chronic inflammation comprises an acute and/or chronic neurogenic inflammation. Acute and/or chronic neurogenic inflammation refers to an inflammatory response initiated and/or maintained through the release of inflammatory molecules like SP or CGRP which released from peripheral sensory nerve terminals (i.e., an efferent function, in contrast to the normal afferent signaling to the spinal cord in these nerves). Acute and/or chronic neurogenic inflammation includes both primary inflammation and secondary neurogenic inflammation. Primary neurogenic inflammation refers to tissue inflammation (inflammatory symptoms) that is initiated by, or results from, the release of substances from primary sensory nerve terminals (such as C and A-delta fibers). Secondary neurogenic inflammation refers to tissue inflammation initiated by non-neuronal sources (e.g., extravasation from vascular bed or tissue interstitium-derived, such as from mast cells or immune cells) of inflammatory mediators, such as peptides or cytokines, stimulating sensory nerve terminals and causing a release of inflammatory mediators from the nerves. The net effect of both forms (primary and secondary) of acute and/or chronic neurogenic inflammation is to have an inflammatory state that is maintained by the sensitization of the peripheral sensory nerve fibers. The physiological consequence of the resulting acute and/or chronic neurogenic inflammation depends on the tissue in question, producing, such as, e.g., cutaneous pain (allodynia, hyperalgesia), joint pain and/or arthritis, visceral pain and dysfunction, pulmonary dysfunction (asthma, COPD), and bladder dysfunction (pain, overactive bladder).

### Conclusion

Here we report, using multiple primary human MSC lines, a functional and biochemical assessment of two distinct approaches using the alpha (1,3)-fucosyltransferase FUT6 for transiently increasing cell surface E-selectin ligands, and their impact on MSC homing to bone. This study represents the first direct comparison between intracellular and extracellular fucosylation using the same enzyme in a clinically relevant experimental model. Compared to untreated MSCs, both intracellular and extracellular fucosylation markedly increased cell surface E-selectin ligands and improved osteotropism in all primary MSC lines tested, indicating that these approaches are consistent and relevant across multiple MSC donors. Notably, at 24 hours post-transplant, overall osteotropism and levels of extravasation were significantly higher with intracellular than extracellular fucosylation. This finding is likely a reflection of the more sustained expression and increased diversity of cell surface E-selectin ligands on the intracellularly versus extracellularly fucosylated MSCs. Collectively, these results indicate that this simple and non-permanent strategy to enforce fucosylation could be of use in augmenting homing of transplanted MSCs.

### DOCUMENTS CITED

Ankrum J, Karp J M. Mesenchymal stem cell therapy: Two steps forward, one step back. Trends Mol Med. 2010;16:203-209.
Borsig L, Katopodis A G, Bowen B R et al. Trafficking and localization studies of recombinant alpha1, 3-fucosyltransferase VI stably expressed in CHO cells. Glycobiology. 1998;8:259-268.
Cheng H, Byrska-Bishop M, Zhang C T et al. Stem cell membrane engineering for cell rolling using peptide conjugation and tuning of cell-selectin interaction kinetics. Biomaterials. 2012;33:5004-5012.
Dimitroff C J, Lee J Y, Rafii S et al. Cd44 Is a Major E-Selectin Ligand on Human Hematopoietic Progenitor Cells. The Journal of Cell Biology. 2001;153:1277-1286.
Dominici M, Le Blanc K, Mueller I et al. Minimal criteria for defining multipotent mesenchymal stromal cells. The International Society for Cellular Therapy position statement. Cytotherapy. 2006;8:315-317.
Galipeau J. The mesenchymal stromal cells dilemma—does a negative phase III trial of random donor mesenchymal stromal cells in steroid-resistant graft-versus-host disease represent a death knell or a bump in the road? Cytotherapy. 2013;15:2-8.
Griffin M D, Elliman S J, Cahill E et al. Adult Mesenchymal Stromal Cell Therapy for Inflammatory Diseases: How Well are We Joining the Dots? Stem Cells. 2013.
Hamidouche Z, Fromigue O, Ringe J et al. Priming integrin alpha5 promotes human mesenchymal stromal cell osteoblast differentiation and osteogenesis. Proc Natl Acad Sci USA. 2009;106:18587-18591.
Jones G N, Moschidou D, Lay K et al. Upregulating CXCR4 in human fetal mesenchymal stem cells enhances engraftment and bone mechanics in a mouse model of osteogenesis imperfecta. Stem Cells Transl Med. 2012;1:70-78.
Kumar S, Ponnazhagan S. Bone homing of mesenchymal stem cells by ectopic alpha 4 integrin expression. FASEB J. 2007;21:3917-3927.
Lee R H, Pulin A A, Seo M J et al. Intravenous hMSCs improve myocardial infarction in mice because cells embolized in lung are activated to secrete the anti-inflammatory protein TSG-6. Cell Stem Cell. 2009;5:54-63.
Levy O, Zhao W, Mortensen L J et al. mRNA-engineered mesenchymal stem cells for targeted delivery of interleukin-10 to sites of inflammation. Blood. 2013;122:e22-e32.
Liao W, Pham V, Liu L et al. Mesenchymal stem cells engineered to express selectin ligands and IL-10 exert enhanced therapeutic efficacy in murine experimental autoimmune encephalomyelitis. Biomaterials. 2016;77:87-97.
Lo CY, Antonopoulos A, Dell A et al. The use of surface immobilization of P-selectin glycoprotein ligand-1 on mesenchymal stem cells to facilitate selectin mediated cell tethering and rolling. Biomaterials. 2013;34:8213-8222.
Mandal P K, Rossi D J. Reprogramming human fibroblasts to pluripotency using modified mRNA. Nat Protoc. 2013;8:568-582.
Merzaban J S, Imitola J, Starossom S C et al. Cell surface glycan engineering of neural stem cells augments neurotropism and improves recovery in a murine model of multiple sclerosis. Glycobiology. 2015;25:1392-1409.
Mortensen L J, Levy O, Phillips J P et al. Quantification of Mesenchymal Stem Cell (MSC) delivery to a target site using in vivo confocal microscopy. PIoS one. 2013;8:e78145.
Parmar S, Liu X, Najjar A et al. Ex vivo fucosylation of third-party human regulatory T cells enhances anti-graft-versus-host disease potency in vivo. Blood. 2015;125:1502-1506.
Popat U, Mehta R S, Rezvani K et al. Enforced fucosylation of cord blood hematopoietic cells accelerates neutrophil and platelet engraftment after transplantation. Blood. 2015;125:2885-2892.
Sackstein R, Merzaban J S, Cain D W et al. Ex vivo glycan engineering of CD44 programs human multipotent mesenchymal stromal cell trafficking to bone. Nature Medicine. 2008;14:181-187.
Sackstein R. Glycosyltransferase-programmed stereosubstitution (GPS) to create HCELL: engineering a roadmap for cell migration. Immunol Rev. 2009;230:51-74.
Sackstein R. Glycosyltransferase-programmed stereosubstitution (GPS) to create HCELL: engineering a roadmap for cell migration. Immunological Reviews. 2009;230:51-74.
Sarkar D, Spencer J A, Phillips J A et al. Engineered cell homing. Blood. 2011;118:e184-191.
Schrepfer S, Deuse T, Reichenspurner H et al. Stem cell transplantation: the lung barrier. Transplant Proc. 2007;39:573-576.
Schweitzer K M, Dräger A M, van der Valk P et al. Constitutive expression of E-selectin and vascular cell adhesion molecule-1 on endothelial cells of hematopoietic tissues. The American Journal of Pathology. 1996;148:165-175.
Shi M, Li J, Liao L et al. Regulation of CXCR4 expression in human mesenchymal stem cells by cytokine treatment: role in homing efficiency in NOD/SCID mice. Haematologica. 2007;92:897-904.
Sipkins D A, Wei X, Wu J W et al. In vivo imaging of specialized bone marrow endothelial microdomains for tumour engraftment. Nature. 2005;435:969-973.
Srouji S, Ben-David D, Fromigue O et al. Lentiviral-mediated integrin alpha5 expression in human adult mesenchymal stromal cells promotes bone repair in mouse cranial and long-bone defects. Hum Gene Ther. 2012;23:167-172.
Wan X, Sato H, Miyaji H et al. Fucosyltransferase VII improves the function of selectin ligands on cord blood hematopoietic stem cells. Glycobiology. 2013;23:1184-1191.
Warren L, Manos PD, Ahfeldt T et al. Highly efficient reprogramming to pluripotency and directed differentiation of human cells with synthetic modified mRNA. Cell Stem Cell. 2010;7:618-630.
Wynn R F, Hart C A, Corradi-Perini C et al. A small proportion of mesenchymal stem cells strongly expresses functionally active CXCR4 receptor capable of promoting migration to bone marrow. Blood. 2004;104:2643-2645.
Xia L, McDaniel J M, Yago T et al. Surface fucosylation of human cord blood cells augments binding to P-selectin and E-selectin and enhances engraftment in bone marrow. Blood. 2004;104:3091-3096.
Yao W, Guan M, Jia J et al. Reversing bone loss by directing mesenchymal stem cells to bone. Stem Cells. 2013;31:2003-2014.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

1. A method of enforcing expression of an E-selectin and/or L-selectin ligand on a surface of a cell, the method comprising the steps of:
providing to the cell a nucleic acid encoding a glycosyltransferase, and
culturing the cell under conditions sufficient to express the glycosyltransferase, wherein the expressed glycosyltransferase modifies a terminal sialylated lactosamine present on a glycoprotein of the cell to enforce expression the E-selectin and/or L-selectin ligand.

2. The method of claim 1, wherein the glycosyltransferase is an alpha 1,3-fucosyltransferase.

3. The method of claim 2, wherein the alpha 1,3-fucosyltransferase is alpha 1,3-fucosyltransferase FTIII, FTIV, FTV, FTVI, FTVII, and combinations thereof.

4. The method of claim 2, wherein the glycosyltransferase modifies the terminal sialylated lactosamine intracellularly.

5. A method of enabling and/or increasing binding of a cell to E-selectin and/or L-selectin, the method comprising the steps of:
providing to the cell a nucleic acid encoding an alpha 1,3-fucosyltransferase, and
culturing the cell under conditions sufficient for expression of the alpha 1,3-fucosyltransferase by the cell;
wherein the alpha 1,3-fucosyltransferase modifies a glycan chain present on a glycoprotein to create an E-selectin and/or L-selectin ligand and thereby enable and/or increase the binding of the cell to E-selectin and/or L-selectin.

6. The method of claim 5, wherein the cell is a mammalian cell.

7. The method of claim 6, wherein the mammalian cell is a human cell.

8. The method of claim 5, wherein the cell is a stem cell.

9. The method of claim 8, wherein the stem cell is selected from the group consisting of embryonic stem cells, adult stem cells, hematopoietic stem cells and induced pluripotent stem cells (iPSCs).

10. The method of claim 9, wherein the adult stem cell is a mesenchymal stem cell.

11. The method of claim 5, wherein the nucleic acid is provided to the cell by transfection.

12. The method of claim 5, wherein the nucleic acid is provided to the cell by transduction.

13. The method of claim 5, wherein the nucleic acid is selected from the group consisting of a DNA, an RNA, a DNA/RNA hybrid, a cDNA, an mRNA, modified versions thereof, and combinations thereof.

14. The method of claim 13, wherein the nucleic acid is a modified RNA.

15. The method of claim 14, wherein the modified RNA is modRNA.

16. The method of claim 5, wherein the alpha 1,3-fucosyltransferase is a human alpha 1,3-fucosyltransferase.

17. The method of claim 5, wherein the alpha 1,3-fucosyltransferase is human FTVI.

18. The method of claim 5, wherein the alpha 1,3-fucosyltransferase fucosylates a glycoprotein selected from the group consisting of PSGL-1, CD43, CD44, and combinations thereof.

19. A method of increasing homing and/or extravasation in a population of cells transplanted into a subject, the method comprising the steps of:
providing to the population of cells a nucleic acid encoding an alpha 1,3-fucosyltransferase,
culturing the population of cells under conditions sufficient for expression of the alpha 1,3-fucosyltransferase by one or more modified cells within the population, wherein the alpha 1,3-fucosyltransferase fucosylates a glycan chain present on a glycoprotein to create modified cells in which E-selection and/or L-selectin ligand expression is enforced; and
transplanting the population of cells into the subject, wherein the modified cells having enforced E-selectin and/or L-selectin ligand expression display increased homing and/or extravasation to therapeutically useful sites.

20. The method of claim 19, wherein the population of cells is a population of mammalian cells.

21. The method of claim 20, wherein the population of cells is a population of human cells.

22. The method of claim 19, wherein the population of mammalian cells is a population of stem cells.

23. The method of claim 22, wherein the population of stem cells is selected from the group consisting of embryonic stem cells, adult stem cells, hematopoietic stem cells and induced pluripotent stem cells (iPSCs).

24. The method of claim 23, wherein the adult stem cells are mesenchymal stem cells.

25. The method of claim 19, wherein the nucleic acid is provided to the population of cells by transfection.

26. The method of claim 19, wherein the nucleic acid is provided to the population of cells by transduction.

27. The method of claim 19, wherein the nucleic acid is selected from the group consisting of a DNA, an RNA, a DNA/RNA hybrid, a cDNA, an mRNA, modified versions thereof, and combinations thereof.

28. The method of claim 19, wherein the nucleic acid is a modified RNA.

29. The method of claim 28, wherein the modified RNA is modRNA.

30. The method of claim 19, wherein the alpha 1,3-fucosyltransferase is a human alpha 1,3-fucosyltransferase.

31. The method of claim 19, wherein the alpha 1,3-fucosyltransferase is human FTVI.

32. The method of claim 19, wherein the alpha 1,3-fucosyltransferase fucosylates a glycoprotein selected from the group consisting of PSGL-1, CD43, CD44, and combinations thereof.

33. The method of claim 19, wherein the step of transplanting occurs intravenously.

34. The method of claim 19, wherein the step of transplanting occurs near the site of desired extravasation.

35. A method of producing modified cells for transplanting into a subject in need thereof, the method comprising the steps of:
obtaining a population of cells to be modified;
providing to the population of cells a nucleic acid encoding an alpha 1,3-fucosyltransferase; and
culturing the population of cells under conditions sufficient for expression of the alpha 1,3-fucosyltransferase by one or more modified cells within the population, wherein the alpha 1,3-fucosyltransferase modifies a glycan chain present on a glycoprotein to create an E-selectin and/or L-selectin ligand.

36. The method of claim 35, wherein the population of cells is a population of mammalian cells.

37. The method of claim 36, wherein the population of mammalian cells is a population of human cells.

38. The method of claim 35, wherein the population of cells is a population of stem cells.

39. The method of claim 38, wherein the population of stem cells is selected from the group consisting of embryonic stem cells, adult stem cells, hematopoietic stem cells and induced pluripotent stem cells (iPSCs).

40. The method of claim 39, wherein the adult stem cells are mesenchymal stem cells.

41. The method of claim 35, wherein the nucleic acid is provided to the population of cells by transfection.

42. The method of claim 35, wherein the nucleic acid is provided to the population of cells by transduction.

43. The method of claim 35, wherein the alpha 1,3-fucosyltransferase is a human alpha 1,3-fucosyltransferase.

44. The method of claim 35, wherein the alpha 1,3-fucosyltransferase is human FTVI.

45. The method of claim 35, wherein the alpha 1,3-fucosyltransferase fucoylates a glycoprotein selected from the group consisting of PSGL-1, CD43, CD44, and combinations thereof.

46. A method of producing modified stem cells for transplanting into a subject, the method comprising the steps of:
obtaining a population of stem cells to be modified;
providing to the population of stem cells a cDNA or modified RNA encoding an alpha 1,3-fucosyltransferase; and
culturing the population of stem cells under conditions sufficient for expression of the alpha 1,3-fucosyltransferase by one or more modified cells within the population, wherein the expressed alpha 1,3-fucosyltransferase fucosylates CD44 present on or in the one or more modified cells.

47. The method of claim 46, wherein the alpha 1,3-fucosyltransferase is human FTVI.

48. The method of claim 46, wherein the stem cells are human stem cells.

49. The method of claim 48, wherein the human stem cells are selected from the group consisting of embryonic stem cells, adult stem cells, hematopoietic stem cells and induced pluripotent stem cells (iPSCs).

50. The method of claim 49, wherein the adult stem cells are mesenchymal stem cells.

51. The method of claim 46, wherein the cDNA or modified RNA is provided by transduction.

52. The method of claim 51, wherein the modified RNA is modRNA.

53. The method of any one of claims 1-52, further comprising the step of carrying out extracellular fucosylation of CD44 on the surface of the stem cells.

54. A method of treating or ameliorating the effects of a symptom, a disease or an injury in a subject in need thereof, the method comprising the steps of:
obtaining a population of cells produced by the method of any one of claims 35-53; and
transplanting an effective amount of the population of cells into the subject, wherein the transplanted cells extravasate to a site expressing E-selectin and/or L-selectin so as thereby to treat or ameliorate the effects of the symptom, disease or injury in the subject.

55. The method of claim 54, wherein the disease is selected from the group consisting of an inflammatory disorder, an autoimmune disease, a degenerative disease, cardiovascular disease, ischemic disease, cancer, a genetic disease, a metabolic disorder and an idiopathic disorder.

56. The method of claim 54, wherein the injury is selected from the group consisting of a physical injury, adverse drug effects, toxic injury, and an iatrogenic condition.

57. The method of claim 54, wherein the subject is a mammal.

58. The method of claim 57, wherein the mammal is selected from the group consisting of humans, primates, farm animals, and domestic animals.

59. The method of claim 58, wherein the mammal is human.

60. The method of claim 54, wherein the transplanting occurs intravenously.

61. The method of claim 54, wherein the transplanting occurs near the site of desired extravasation.

62. The method of claim 61, wherein the site of desired extravasation is the bone marrow.

63. The method of claim 61, wherein the site of desired extravasation is the site of an injury or inflammation.

64. A pharmaceutical composition comprising a population of cells produced by the method of claim 35 and a pharmaceutically acceptable carrier.

65. A kit for treating or ameliorating the effects of a symptom, a disease or an injury in a subject in need thereof comprising the composition of claim 64, packaged together with instructions for its use.

66. A method for inducing and/or enhancing homing of a population of cells to a therapeutic target in a subject in need thereof, the method comprising:
(a) providing to the population of cells a nucleic acid encoding a polypeptide, which enforces transient expression of a ligand that binds to a receptor at the therapeutic target; and
(b) allowing the population of cells to express the polypeptide, wherein upon expression of the polypeptide homing of one or more cells in the population to a therapeutic target is induced and/or enhanced.

67. The method according to claim 66, wherein the population of cells is selected from the group consisting of stem cells, tissue progenitor cells, antigen-specific T-cells, T-regulator cells, antigen-pulsed dendritic cells, NK cells, NKT cells, and leukocytes.

68. The method according to claim 67, wherein the population of cells are T-lymphocytes.

69. The method according to claim 67, wherein the population of cells are chimeric antigen receptor T-cells.

70. The method according to claim 66, wherein the population of cells is culture-expanded prior to step (a).

71. The method according to claim 66, wherein the therapeutic target is a tumor.


SEQUENCE LISTING (10 sequences)

SEQ ID NO: 1 - DNA, 2043 bp, Homo sapiens
aggaaacctgccatggcctcctggtgagctgtcctcatccactgctcgctgcctctccagatactctgacccatggatcccctgggtgcagccaagccacaatggccatggcgccgctgtctggccgcactgctatttcagctgctggtggctgtgtgtttcttctcctacctgcgtgtgtcccgagacgatgccactggatcccctagggctcccagtgggtcctcccgacaggacaccactcccacccgccccaccctcctgatcctgctatggacatggcctttccacatccctgtggctctgtcccgctgttcagagatggtgcccggcacagccgactgccacatcactgccgaccgcaaggtgtacccacaggcagacacggtcatcgtgcaccactgggatatcatgtccaaccctaagtcacgcctcccaccttccccgaggccgcaggggcagcgctggatctggttcaacttggagccaccccctaactgccagcacctggaagccctggacagatacttcaatctcaccatgtcctaccgcagcgactccgacatcttcacgccctacggctggctggagccgtggtccggccagcctgcccacccaccgctcaacctctcggccaagaccgagctggtggcctgggcggtgtccaactggaagccggactcagccagggtgcgctactaccagagcctgcaggctcatctcaaggtggacgtgtacggacgctcccacaagcccctgcccaaggggaccatgatggagacgctgtcccggtacaagttctacctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactggatttctgcaaggcctgctggaaactgcagcaggaatccaggtaccagacggtgcgcagcatagcggcttggttcacctgagaggccggcatggtgcctgggctgccgggaacctcatctgcctggggcctcacctgctggagtcctttgtggccaaccctctctcttacctgggacctcacacgctgggcttcacggctgccaggagcctctcccctccagaagacttgcctgctagggacctcgcctgctggggacctcgcctgttggggacctcacctgctggggacctcacctgctggggaccttggctgctggaggctgcacctactgaggatgtcggcggtcggggactttacctgctgggacctgctcccagagaccttgccacactgaatctcacctgctggggacctcaccctggagggccctgggccctggggaactggcttacttggggccccacccgggagtgatggttctggctgatttgtttgtgatgttgttagccgcctgtgaggggtgcagagagatcatcacggcacggtttccagatgtaatactgcaaggaaaaatgatgacgtgtctcctcactctagaggggttggtcccatgggttaagagctcaccccaggttctcacctcaggggttaagagctcagagttcagacaggtccaagttcaagcccaggaccaccacttatagggtacaggtgggatcgactgtaaatgaggacttctggaacattccaaatattctggggttgagggaaattgctgctgtctacaaaatgccaagggtggacaggcgctgtggctcacgcctgtaattccagcactttgggaggctgaggtaggaggattgattgaggccaagagttaaagaccagcctggtcaatatagcaagaccacgtctctaaataaaaaataataggccggccaggaaaaaaaaaaaaaaaaaaaaa

SEQ ID NO: 2 - protein, 361 aa, Homo sapiens
MDPLGAAKPQWPWRRCLAALLFQLLVAVCFFSYLRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 3 - DNA, 2159 bp, Homo sapiens
cgctcctccacgcctgcggacgcgtggcgagcggaggcagcgctgcctgttcgcgccatgggggcaccgtggggctcgccgacggcggcggcgggcgggcggcgcgggtggcgccgaggccgggggctgccatggaccgtctgtgtgctggcggccgccggcttgacgtgtacggcgctgatcacctacgcttgctgggggcagctgccgccgctgccctgggcgtcgccaaccccgtcgcgaccggtgggcgtgctgctgtggtgggagcccttcggggggcgcgatagcgccccgaggccgccccctgactgccggctgcgcttcaacatcagcggctgccgcctgctcaccgaccgcgcgtcctacggagaggctcaggccgtgcttttccaccaccgcgacctcgtgaaggggccccccgactggcccccgccctggggcatccaggcgcacactgccgaggaggtggatctgcgcgtgttggactacgaggaggcagcggcggcggcagaagccctggcgacctccagccccaggcccccgggccagcgctgggtttggatgaacttcgagtcgccctcgcactccccggggctgcgaagcctggcaagtaacctcttcaactggacgctctcctaccgggcggactcggacgtctttgtgccttatggctacctctaccccagaagccaccccggcgacccgccctcaggcctggccccgccactgtccaggaaacaggggctggtggcatgggtggtgagccactgggacgagcgccaggcccgggtccgctactaccaccaactgagccaacatgtgaccgtggacgtgttcggccggggcgggccggggcagccggtgcccgaaattgggctcctgcacacagtggcccgctacaagttctacctggctttcgagaactcgcagcacctggattatatcaccgagaagctctggcgcaacgcgttgctcgctggggcggtgccggtggtgctgggcccagaccgtgccaactacgagcgctttgtgccccgcggcgccttcatccacgtggacgacttcccaagtgcctcctccctggcctcgtacctgcttttcctcgaccgcaaccccgcggtctatcgccgctacttccactggcgccggagctacgctgtccacatcacctccttctgggacgagccttggtgccgggtgtgccaggctgtacagagggctggggaccggcccaagagcatacggaacttggccagctggttcgagcggtgaagccgcgctcccctggaagcgacccaggggaggccaagttgtcagctttttgatcctctactgtgcatctccttgactgccgcatcatgggagtaagttcttcaaacacccatttttgctctatgggaaaaaaacgatttaccaattaatattactcagcacagagatgggggcccggtttccatattttttgcacagctagcaattgggctccctttgctgctgatgggcatcattgtttaggggtgaaggagggggttcttcctcaccttgtaaccagtgcagaaatgaaatagcttagcggcaagaagccgttgaggcggtttcctgaatttccccatctgccacaggccatatttgtggcccgtgcagcttccaaatctcatacacaactgttcccgattcacgtttttctggaccaaggtgaagcaaatttgtggttgtagaaggagccttgttggtggagagtggaaggactgtggctgcaggtgggactttgttgtttggattcctcacagccttggctcctgagaaaggtgaggagggcagtccaagaggggccgctgacttctttcacaagtactatctgttcccctgtcctgtgaatggaagcaaagtgctggattgtccttggaggaaacttaagatgaatacatgcgtgtacctcactttacataagaaatgtattcctgaaaagctgcatttaaatcaagtcccaaattcattgacttaggggagttcagtatttaatgaaaccctatggagaatttatccctttacaatgtgaatagtcatctcctaatttgtttcttctgtctttatgtttttctataacctggattttttaaatcatattaaaattacagatgtgaaaataaaaaaaa

SEQ ID NO: 4 - protein, 530 aa, Homo sapiens
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 5 - DNA, 1310 bp, Homo sapiens
tttatgacaagctgtgtcataaattataacagcttctctcaggacactgtggccaggaagtgggtgatcttccttaatgaccctcactcctctctcctctcttcccagctactctgacccatggatcccctgggcccagccaagccacagtggctgtggcgccgctgtctggccgggctgctgtttcagctgctggtggctgtgtgtttcttctcctacctgcgtgtgtcccgagacgatgccactggatcccctaggccagggcttatggcagtggaacctgtcaccggggctcccaatgggtcccgctgccaggacagcatggcgacccctgcccaccccaccctactgatcctgctgtggacgtggccttttaacacacccgtggctctgccccgctgctcagagatggtgcccggcgcggccgactgcaacatcactgccgactccagtgtgtacccacaggcagacgcggtcatcgtgcaccactgggatatcatgtacaaccccagtgccaacctcccgccccccaccaggccgcaggggcagcgctggatctggttcagcatggagtcccccagcaactgccggcacctggaagccctggacggatacttcaatctcaccatgtcctaccgcagcgactccgacatcttcacgccctacggctggctggagccgtggtccggccagcctgcccacccaccgctcaacctctcggccaagaccgagctggtggcctgggcggtgtccaactggaagccggactcggccagggtgcgctactaccagagcctgcaggctcatctcaaggtggacgtgtacggacgctcccacaagcccctgcccaaggggaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccgcccgacgccttcatccacgtggatgacttccagagccccaaggacctggcccggtacctgcaggagctggacaaggaccacgcccgctacctgagctactttcgctggcgggagacgctgcggcctcgctccttcagctgggcactggctttctgcaaggcctgctggaagctgcagcaggaatccaggtaccagacggtgcgcagcatagcggcttggttcacctgagaggccggcatggggcctgggctgccagggacctcactttcccagggcctcacctacctagggtc

SEQ ID NO: 6 - protein, 374 aa, Homo sapiens
MDPLGPAKPQWLWRRCLAGLLFQLLVAVCFFSYLRVSRDDATGSPRPGLMAVEPVTGAPNGSRCQDSMATPAHPTLLILLWTWPFNTPVALPRCSEMVPGAADCNITADSSVYPQADAVIVHHWDIMYNPSANLPPPTRPQGQRWIWFSMESPSNCRHLEALDGYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQQESRYQTVRSIAAWFT

SEQ ID NO: 7 - DNA, 1126 bp, Homo sapiens
cagatactctgacccatggatcccctgggcccggccaagccacagtggtcgtggcgctgctgtctgaccacgctgctgtttcagctgctgatggctgtgtgtttcttctcctatctgcgtgtgtctcaagacgatcccactgtgtaccctaatgggtcccgcttcccagacagcacagggacccccgcccactccatccccctgatcctgctgtggacgtggccttttaacaaacccatagctctgccccgctgctcagagatggtgcctggcacggctgactgcaacatcactgccgaccgcaaggtgtatccacaggcagacgcggtcatcgtgcaccaccgagaggtcatgtacaaccccagtgcccagctcccacgctccccgaggcggcaggggcagcgatggatctggttcagcatggagtccccaagccactgctggcagctgaaagccatggacggatacttcaatctcaccatgtcctaccgcagcgactccgacatcttcacgccctacggctggctggagccgtggtccggccagcctgcccacccaccgctcaacctctcggccaagaccgagctggtggcctgggcagtgtccaactgggggccaaactccgccagggtgcgctactaccagagcctgcaggcccatctcaaggtggacgtgtacggacgctcccacaagcccctgccccagggaaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccgcccgacgccttcatccacgtggacgacttccagagccccaaggacctggcccggtacctgcaggagctggacaaggaccacgcccgctacctgagctactttcgctggcgggagacgctgcggcctcgctccttcagctgggcactcgctttctgcaaggcctgctggaaactgcaggaggaatccaggtaccagacacgcggcatagcggcttggttcacctgagaggcccggcatggggcctgggctgccaggg

SEQ ID NO: 8 - protein, 359 aa, Homo sapiens
MDPLGPAKPQWSWRCCLTTLLFQLLMAVCFFSYLRVSQDDPTVYPNGSRFPDSTGTPAHSIPLILLWTWPFNKPIALPRCSEMVPGTADCNITADRKVYPQADAVIVHHREVMYNPSAQLPRSPRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 9 - DNA, 1701 bp, Homo sapiens
aaggagcacagttccaggcggggctgagctagggcgtagctgtgatttcaggggcacctctggcggctgccgtgatttgagaatctcgggtctcttggctgactgatcctgggagactgtggatgaataatgctgggcacggccccacccggaggctgcgaggcttgggggtcctggccggggtggctctgctcgctgccctctggctcctgtggctgctggggtcagcccctcggggtaccccggcaccccagcccacgatcaccatccttgtctggcactggcccttcactgaccagcccccagagctgcccagcgacacctgcacccgctacggcatcgcccgctgccacctgagtgccaaccgaagcctgctggccagcgccgacgccgtggtcttccaccaccgcgagctgcagacccggcggtcccacctgcccctggcccagcggccgcgagggcagccctgggtgtgggcctccatggagtctcctagccacacccacggcctcagccacctccgaggcatcttcaactgggtgctgagctaccggcgcgactcggacatctttgtgccctatggccgcctggagccccactgggggccctcgccaccgctgccagccaagagcagggtggccgcctgggtggtcagcaacttccaggagcggcagctgcgtgccaggctgtaccggcagctggcgcctcatctgcgggtggatgtctttggccgtgccaatggacggccactgtgcgccagctgcctggtgcccaccgtggcccagtaccgcttctacctgtcctttgagaactctcagcaccgcgactacattacggagaaattctggcgcaacgcactggtggctggcactgtgccagtggtgctggggcccccacgggccacctatgaggccttcgtgccggctgacgccttcgtgcatgtggatgactttggctcagcccgagagctggcggctttcctcactggcatgaatgagagccgataccaacgcttctttgcctggcgtgacaggctccgcgtgcgactgttcaccgactggcgggaacgtttctgtgccatctgtgaccgctacccacacctaccccgcagccaagtctatgaggaccttgagggttggtttcaggcctgagatccgctggccgggggaggtgggtgtgggtggaagggctgggtgtcgaaatcaaaccaccaggcatccggcccttaccggcaagcagcgggctaacgggaggctgggcacagaggtcaggaagcaggggtggggggtgcaggtgggcactggagcatgcagaggaggtgagagtgggagggaggtaacgggtgcctgctgcggcagacgggaggggaaaggctgccgaggaccctccccaccctgaacaaatcttgggtgggtgaaggcctggctggaagagggtgaaaggcagggcccttggggctggggggcaccccagcctgaagtttgtgggggccaaacctgggaccccgagcttcctcggtagcagaggccctgtggtccccgagacacaggcacgggtccctgccacgtccatagttctgaggtccctgtgtgtaggctggggcggggcccaggagaccacggggagcaaaccagcttgttctgggctcagggagggagggcggtggacaataaacgtctgagcagtgaaaaaaaaaaaaaa

SEQ ID NO: 10 - protein, 342 aa, Homo sapiens
MNNAGHGPTRRLRGLGVLAGVALLAALWLLWLLGSAPRGTPAPQPTITILVWHWPFTDQPPELPSDTCTRYGIARCHLSANRSLLASADAVVFHHRELQTRRSHLPLAQRPRGQPWVWASMESPSHTHGLSHLRGIFNWVLSYRRDSDIFVPYGRLEPHWGPSPPLPAKSRVAAWVVSNFQERQLRARLYRQLAPHLRVDVFGRANGRPLCASCLVPTVAQYRFYLSFENSQHRDYITEKFWRNALVAGTVPVVLGPPRATYEAFVPADAFVHVDDFGSARELAAFLTGMNESRYQRFFAWRDRLRVRLFTDWRERFCAICDRYPHLPRSQVYEDLEGWFQA